(19) United States
(12) Patent Application Publication
(10) Pub. No.: US 2017/0136008 A1
(43) Pub. Date: May 18, 2017

(54) OXABICYCLOHEPTANES AND OXABICYCLOHEPTENES FOR THE TREATMENT OF OVARIAN CANCER

(71) Applicants: John S. Kovach, East Setauket, NY (US); Zhengping Zhuang, Bethesda, MD (US); Ki-eun Chang, Los Angeles, CA (US); Matthew Hall, Damestown, MD (US); Michael M. Gottesman, Bethesda, MD (US)

(72) Inventors: John S. Kovach, East Setauket, NY (US); Zhengping Zhuang, Bethesda, MD (US); Ki-eun Chang, Los Angeles, CA (US); Matthew Hall, Damestown, MD (US); Michael M. Gottesman, Bethesda, MD (US)

(73) Assignees: Lixte Biotechnology, Inc., East Setauket, NY (US); The United States of America, as Represented by th e Secretary, Department of Health & Human Service, Bethesda, MD (US)

(21) Appl. No.: 15/320,153

(22) PCT Filed: Jun. 19, 2015
(86) PCT No.: PCT/US15/36693

§ 371 Date: Dec. 19, 2016

Related U.S. Application Data
(60) Provisional application No. 62/015,095, filed on Jun. 20, 2014.

(51) Int. Cl. A61K 31/496; A61K 31/704; A61K 33/24

(52) U.S. Cl. CPC A61K 31/496; A61K 33/24; A61K 31/704

(57) ABSTRACT

A method of treating ovarian cancer in a subject afflicted therewith comprising administering to the subject an effective amount of an anti-cancer agent and an effective amount of a compound having the structure:

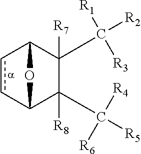

This application claims priority of U.S. Provisional Application No. 62/015,095, filed Jun. 20, 2014, the contents of which are hereby incorporated by reference.

Throughout this application various publications are referenced. The disclosures of these documents in their entireties are hereby incorporated by reference into this application in order to more fully describe the state of the art to which this invention pertains.

### BACKGROUND OF THE INVENTION

Ovarian cancer is the fifth leading cause of cancer death in women, taking the lives of over 14,000 patients in the United States in 2013 (American Cancer Society 2014). Due to non-specific initial symptoms and unreliable screening measures, most patients present with late-stage disease and a poor (less than 20%) chance of long-term survival (Partridge, E. et al. 2009). Current standard of treatment involves maximal debulking at initial surgery followed by combination chemotherapy consisting of a platinum-based compound and a taxane (Armstrong, D. K. et al. 2006). Although most patients have an initial positive response, most eventually develop multidrug resistance and die of progressive cancer (Markman, M. et al. 1991).

Cisplatin [cis-[PtCl₂(NH₃)₂]] is a platinum-based drug that is commonly used in the treatment of ovarian cancer. It is generally believed that cisplatin acts by forming DNA crosslinks that lead to the induction of double strand breaks (DSB) formed as a consequence of the innate repair mechanisms of the cell. The consequent DSB accumulation and stalled DNA fork progression result in apoptosis of sensitive cells (Wang, D. et al. 2005). Despite its high potency, clinical resistance to cisplatin is common, and potential toxicities including nephrotoxicity, nausea/vomiting, neurotoxicity, and ototoxicity limit the effective dose that can be employed (Wong, E. et al. 1999). Platinum resistance in ovarian cancer mainly involves an increase in tolerance and/or repair of the DNA adducts as well as a failure of apoptotic pathway activation (Eliopoulos, A. G. et al. 1995; Mamenta, E. L. et al. 1994; Shen, D. W. et al. 2012). Importantly, greater than 90% of ovarian cancers harbor inactivating mutations of p53 and lack the ability to arrest the cell cycle at the G1/S phase junction (Cancer Genome Atlas Pilot Project 2011; Kanchi, K. L. et al. 2014). Cisplatin thus induces potent S phase and G2/M phase cell cycle arrests, allowing DNA damage repair (Siegel, R. et al. 2012).

The extent of cellular damage and the fidelity of DNA repair following therapeutic intervention are often gauged by the degree of phosphorylation of key intermediaries within the response signaling pathways. It has been shown that constitutive phosphorylation of these intermediaries is abarometer of the critical cellular processes that determine whether the cell will repair the damaged DNA or induce apoptotic cell death (Chowdhury, D. et al. 20005; Lee, D. H. et al. 2011; Martin, S. A. et al. 2005; Clemenson, C. et al. 2009). The DNA damage response is facilitated by a highly integrated and complex series of phosphorylation and dephosphorylation events regulated by key kinases and phosphatases, respectively. For example, the serine/threonine kinases ATM and ATR are activated following double strand break induction or stalled DNA replication fork and are implicated in regulating DNA repair, cell cycle checkpoints, and apoptotic signaling. ATM/ATR directly and indirectly exert these effects by controlling the phosphorylation of downstream target proteins such as BRCA1, H2AX, Chk1, and Chk2 (Clemenson, C. et al. 2009). Increased and constitutive phosphorylation of numerous other non-ATM/ATR pathway signaling proteins have also been observed following cisplatin treatment and may be correlated with the extent of apoptotic induction. For example, sustained SAPK/JNK (stress-activated protein kinase/c-Jun N-terminal kinase) activation following cisplatin treatment plays a role in both extrinsic and mitochondrial apoptosis (Mansouri, A. et al. 2003).

Protein phosphatase 2A (PP2A) is a ubiquitous serine/threonine phosphatase that dephosphorylates numerous proteins of both ATM/ATR-dependent and -independent response pathways (Mumby, M. 2007). Pharmacologic inhibition of PP2A has previously been shown to sensitize cancer cells to radiation-mediated DNA damage via constitutive phosphorylation of various signaling proteins, such as p53, γH2AX, PLK1 and Akt, resulting in cell cycle deregulation, inhibition of DNA repair, and apoptosis (We, D. et al. 2013).

Cantharidin, the principle active ingredient of blister beetle extract (Mylabris), is a compound derived from traditional Chinese medicine that has been shown to be a potent inhibitor of PP2A (Efferth, T. et al. 2005). Although cantharadin has previously been used in the treatment of hepatomas and has shown efficacy against multidrug-resistant leukemia cell lines (Efferth, T. et al. 2002), its severe toxicity limits its clinical usefulness. LB100 is a small molecule derivative of cantharadin with significantly less toxicity (Kovach, J. 2012). Previous pre-clinical studies have shown that LB100 can enhance the cytotoxic effects of temozolomide, doxorubicin, and radiation therapy against glioblastoma (GBM), metastatic pheochromocytoma, and pancreatic cancer (Wei, D. et al. 2013; Lu, J. et al. 2009; Zhang, C. et al. 2010; Martiniova, L. et al. 2011). LB100 is also undergoing a phase 1 study in combination with docetaxel for the treatment of solid tumors (Chung, V. 2013).

### SUMMARY OF THE INVENTION

The present invention provides a method of treating ovarian cancer in a subject afflicted therewith comprising administering to the subject an effective amount of an anti-cancer agent and an effective amount of a compound having the structure:

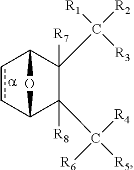

wherein
bond α is present or absent;
R₁ and R₂ together are ═O;
R₃ is OH, O⁻, OR₉, O(CH₂)₁₋₆R₉, SH, S⁻, or SR₉,
   wherein R₉ is H, alkyl, alkenyl, alkynyl or aryl;
R₄ is

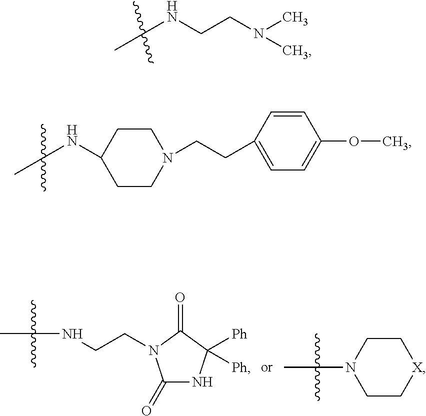

where X is O, S, NR₁₀, N⁺HR₁₀ or N⁺R₁₀R₁₀,
   where each R₁₀ is independently H, alkyl, alkenyl, alkynyl, aryl,

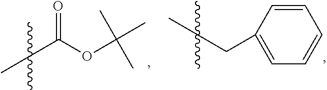

—CH₂CN, —CH₂CO₂R₁₁, or —CH₂COR₁₁,
   wherein each R₁₁ is independently H, alkyl, alkenyl or alkynyl;
R₅ and R₆ taken together are ═O;
R₇ and Re are each H,
   or a salt, zwitterion, or ester thereof,
   so as to thereby treat the ovarian cancer in the subject.

The present invention also provides a method of treating ovarian cancer in a subject afflicted therewith comprising administering to the subject an effective amount of an anti-cancer agent and an effective amount of a compound having the structure:

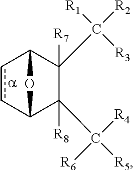

wherein
bond α is present or absent;
R₁ and R₂ together are ═O;
R₃ is OH, O⁻, OR₉, O(CH₂)₁₋₆R₉, SH, S⁻, or SR₉,
   wherein R₉ is H, alkyl, alkenyl, alkynyl or aryl;
R₄ is

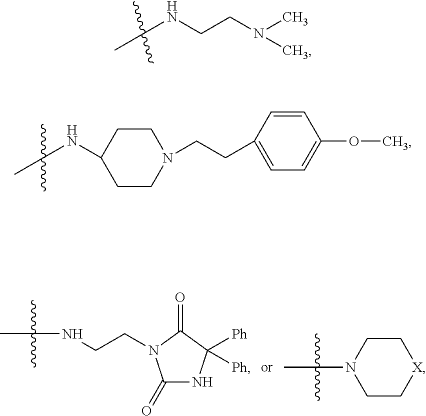

where X is O, S, NR₁₀, N⁺HR₁₀ or N⁺R₁₀R₁₀,
   where each R₁₀ is independently H, alkyl, alkenyl, alkynyl, aryl,

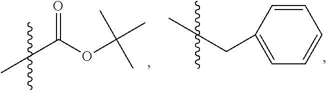

—CH₂CN, —CH₂CO₂R₁₁, or —CH₂COR₁₁,
   wherein each R₁₁ is independently H, alkyl, alkenyl or alkynyl;
R₅ and R₆ taken together are ═O;
R₇ and R₈ are each H,
   or a salt, zwitterion, or ester thereof,
   so as to thereby treat the ovarian cancer in the subject, wherein the ovarian cancer is resistant to the anti-cancer agent or at least one other anti-cancer agent.

The present invention further provides a method of reducing the likelihood of a subject afflicted with ovarian cancer developing drug resistance to an anti-cancer agent comprising administering to the subject an effective amount of a compound having the structure:

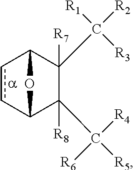

wherein
bond α is present or absent;
R₁ and R₂ together are ═O;
R₃ is OH, O⁻, OR₉, O(CH₂)₁₋₆R₉, SH, S⁻, or SR₉,
   wherein R₉ is H, alkyl, alkenyl, alkynyl or aryl;
R₄ is

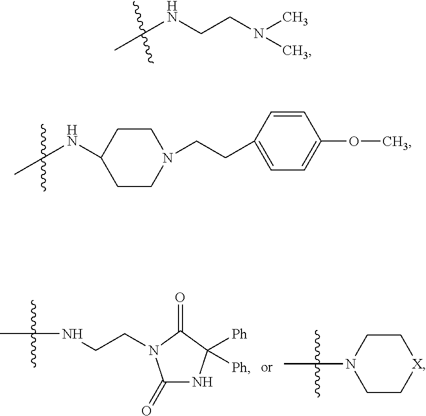

where X is O, S, NR₁₀, N⁺HR₁₀ or N⁺R₁₀R₁₀,
   where each R₁₀ is independently H, alkyl, alkenyl, alkynyl, aryl,

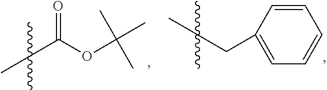

—CH₂CN, —CH₂CO₂R₁₁, or —CH₂COR₁₁,
   wherein each R₁₁ is independently H, alkyl, alkenyl or alkynyl;
R₅ and R₆ taken together are ═O;
R₇ and R₉ are each H,
   or a salt, zwitterion, or ester thereof,
   and administering an effective amount of the anti-cancer agent so as to thereby reduce the likelihood of the subject afflicted with the ovarian cancer developing drug resistance to the anti-cancer agent.

### BRIEF DESCRIPTION OF THE FIGURES

FIG. 1: Inhibition of PP2A by LB100 sensitizes ovarian cancer cells to cisplatin cytotoxicity. A, MTT assay showing increased cytotoxicity in SKOV-3 cells (B) and OVCAR-8 cells (C) for both IC₂₅ and IC₇₅ doses of cisplatin when cells were pre-treated with LB100 compared to either drug alone. D, Western blots following 48 hr treatment shows apoptosis via cleaved PARP and cleaved caspase 3. E, Western blot following 72 hr treatment with two different concentrations of cisplatin shows that pre-treatment with LB100 enhances apoptosis induced with a sub-lethal dose (IC₂₅) of cisplatin.

FIG. 2: Potential mechanism of cisplatin sensitization induced by LB100. A, LB100 sensitizes ovarian cancer cells by constitutive hyperphosphorylation of DNA damage response proteins and the JNK/MAPK pathway in SKOV-3 cells. LB100 alone or in combination with cisplatin enhances phosphorylation of Chk2, BRCA1, and JNK without significantly altering the total protein level and this effect was observed over a 72 hr period. B, Sensitization with LB100 allows bypass of cell cycle checkpoints despite DNA damage. Western blot showing reduced Wee1 expression following combination treatment (24 h) and consequent decreased phosphorylation of cdc2, allowing cell cycle progression into the mitotic phase as indicated by p-histone H3 expression. C, LB100 modulates phospho-(Ser) binding motif. Western blot of SKOV-3 blot showing differential expression of p-Ser binding motif when treated with LB100 alone or in combination with two different concentrations of cisplatin.

FIG. 3: Validation of PP2Ac as a mediator of ciplatin cytotoxicity. A, Western blot showing stable knockdown of PP2Ac in OVCAR-8 cells. B, Cell viability (MTT) assay demonstrating increased sensitivity to cisplatin and LB100 following PP2Ac knockdown. C,D, Western Blot showing hyperphosphorylation of Chk1 (S345) for up to 8 hrs following cisplatin washout in PP2Ac knockdown (C) and LB100 treated (D) OVCAR-8 cells.

FIG. 4: LB100 sensitized SKOV-3 intraperitoneal xenografts to the cytotoxic effects of cisplatin. Mice bearing SKOV-3 intraperitoneal metastatic tumors were treated with PBS (vehicle control) (n=4), LB100 (1.5 mg/kg) (n=5), cisplatin (1.5 mg/kg) (n=5), or LB100 (1.5 mg/kg 1 hr pre-cisplatin)+cisplatin (1.5 mg/kg)(n=5) for 6 session given every other day. A, No significant difference in body weight indicates minimal toxicity. B, LB100+cisplatin combination treatment significantly slows tumor growth, as measured by bioluminescence signaling, compared to other treatment groups. Data is represented as mean±SD of relative total photon flux compared to day 1 of treatment. C, Representative imaging of each treatment group. An average (left) and best (right) responder in the combination group is shown by comparison. D, ex-vivo imaging confirms that the signal obtained originates from tumor cells. E, Western blot obtained from ex-vivo tumor samples illustrates hyperphosphorylation of γH2AX, BRCA-1, Chk-1.

FIG. 5: IC₅₀s of transfected lines with parental (non-transporter-expressing) cells or in the presence of an inhibitor (tariquidar). A, parental and ABCG2. B, P-glycoprotein and MRP1/ABCC1.

FIG. 6: Inhibition of cisplatin-resistant KB-CP.5 cells and KB-3-1 human adenocarcinoma cells by LB100 or cisplatin.

FIG. 7: A, Cell viability (MTT) assay demonstrating cytotoxicity of LB100, cisplatin and LB100/cisplatin combinations in PEO-1m ovarian cancer cell line. B, Cell viability (MTT) assay demonstrating cyctoxicicty of LB100, cisplatin and LB100/cisplatin combinations in PEO-1s ovarian cancer cell line. C, Cell viability (MTT) assay demonstrating cyctoxicicty of LB100, cisplatin and LB100/cisplatin combinations in PEO-6 ovarian cancer cell line.

FIG. 8: A, Western blot showing reduced Wee1 expression following combination treatment (24 h) and consequent decreased phosphorylation of cdc2. B, Western Blot showing hyperphosphorylation of Chk1 (S317) for up to 8 hrs following cisplatin washout in LB100 treated cells/35

FIG. 9: A, G2.M regulation pathway in the absence of LB100. B, G2.M regulation pathway in the presence of LB100.

### DETAILED DESCRIPTION OF THE INVENTION

The present invention provides a method of treating ovarian cancer in a subject afflicted therewith comprising administering to the subject an effective amount of an anti-cancer agent and an effective amount of a compound having the structure:

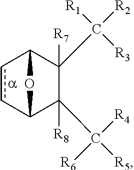

wherein
bond α is present or absent;
R₁ and R₂ together are ═O;
R₃ is OH, O⁻, OR₉, O(CH₂)₁₋₆R₉, SH, S⁻, or SR₉,
   wherein R₉ is H, alkyl, alkenyl, alkynyl or aryl;
R₄ is

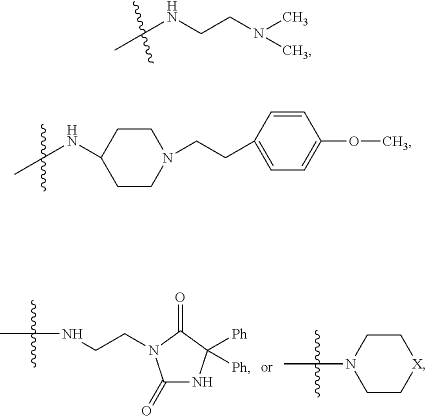

where X is O, S, NR₁₀, N⁺HR₁₀ or N⁺R₁₀R₁₀,
   where each R₁₀ is independently H, alkyl, alkenyl, alkynyl, aryl,

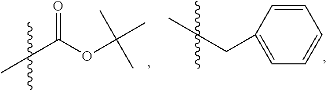

—CH₂CN, —CH₂CO₂R₁₁, or —CH₂COR₁₁,
   wherein each R₁₁ is independently H, alkyl, alkenyl or alkynyl;
R₅ and R₆ taken together are ═O;
R₇ and R₉ are each H,
   or a salt, zwitterion, or ester thereof,
   so as to thereby treat the ovarian cancer in the subject.

The present invention provides also provides a method of treating ovarian cancer in a subject afflicted therewith comprising administering to the subject an effective amount of an anti-cancer agent and an effective amount of a compound having the structure:

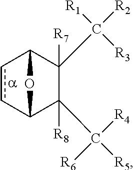

wherein
bond α is present or absent;
R₁ and R₂ together are ═O;
R₃ is OH, O⁻, OR₉, O(CH₂)₁₋₆R₉, SH, S⁻, or SR₉,
   wherein R₉ is H, alkyl, alkenyl, alkynyl or aryl;
R₄ is

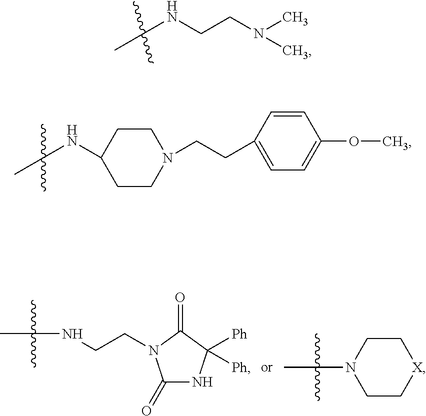

where X is O, S, NR₁₀, N⁺HR₁₀ or N⁺R₁₀R₁₀,
   where each R₁₀ is independently H, alkyl, alkenyl, alkynyl, aryl,

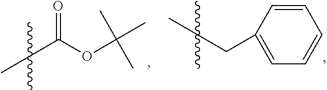

—CH₂CN, —CH₂CO₂R₁₁, or —CH₂COR₁₁,
   wherein each R₁₀ is independently H, alkyl, alkenyl or alkynyl;
R₅ and R₆ taken together are ═O;
R₇ and R₈ are each H,
   or a salt, zwitterion, or ester thereof,
   so as to thereby treat the ovarian cancer in the subject, wherein the ovarian cancer is resistant to the anti-cancer agent or at least one other anti-cancer agent.

In some embodiments, the ovarian cancer in the subject was previously treated with the anti-cancer agent or at least one other anti-cancer agent.

In some embodiments, the amount of the compound and the amount of the anti-cancer agent are each periodically administered to the subject

In some embodiments, the amount of the compound and the amount of the anti-cancer agent are administered simultaneously, separately or sequentially.

In some embodiments, the method comprising administering to the subject an effective amount of the compound and subsequently administering to the subject, after an interval comprising at least 1 hour, the anti-cancer agent.

In some embodiments, the amount of the compound and the amount of the anti-cancer agent when taken together is more effective to treat the subject than when the anti-cancer agent is administered alone.

In some embodiments, the amount of the compound and the amount of the anti-cancer agent when taken together has a greater than additive effect on the ovarian cancer in the subject.

In some embodiments, the amount of the compound and the amount of the anti-cancer agent when taken together is effective to reduce a clinical symptom of the ovarian cancer in the subject.

In some embodiments, the treating comprises inhibiting proliferation of ovarian cancer cells in the subject, inducing apoptosis of ovarian cancer cells in the subject, or reducing the size of an ovarian tumor in the subject.

In some embodiments, the compound enhances the chemotherapeutic effect of the anti-cancer agent.

In some embodiments, the compound enhances delivery of the anti-cancer agent to ovarian cancer cells in the subject.

In some embodiments, the compound increases the concentration of the anti-cancer agent in ovarian cancer in the subject.

In some embodiments, the compound increases blood supply to ovarian cancer cells in the subject thereby enhancing delivery of the anti-cancer agent to the ovarian cancer cells.

In some embodiments, the compound chemosensitizes the ovarian cancer to the anti-cancer agent.

In some embodiments, the compound increases chemosensitization of the ovarian cancer to the anti-cancer agent.

In some embodiments, the compound reduces the resistance of the ovarian cancer to the anti-cancer agent.

In some embodiments, the compound re-sensitizes the ovarian cancer to the anti-cancer agent.

The present invention further provides a method of reducing the likelihood of a subject afflicted with ovarian cancer developing drug resistance to an anti-cancer agent comprising administering to the subject an effective amount of a compound having the structure:

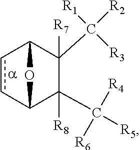

wherein
bond α is present or absent;
R₁ and R₂ together are ═O;
R₃ is OH, O⁻, OR₉, O(CH₂)₁₋₆R₉, SH, S⁻, or SR₉,
   wherein R₉ is H, alkyl, alkenyl, alkynyl or aryl;
R₄ is

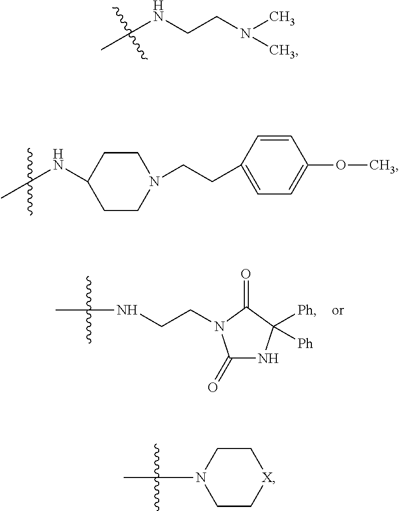

where X is O, S, NR₁₀, N⁺HR₁₀ or N⁺R₁₀R₁₀,
   where each R₁₀ is independently H, alkyl, alkenyl, alkynyl, aryl,

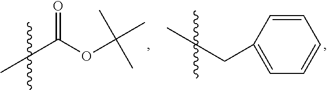

—CH₂CN, —CH₂CO₂R₁₁, or —CH₂COR₁₁,
   wherein each R₁₁ is independently H, alkyl, alkenyl or alkynyl;
R₅ and R₆ taken together are ═O;
R₇ and R₆ are each H,
   or a salt, zwitterion, or ester thereof,
   and administering an effective amount of the anti-cancer agent so as to thereby reduce the likelihood of the subject afflicted with the ovarian cancer developing drug resistance to the anti-cancer agent.

In some embodiments, the ovarian cancer in the subject was previously treated with the anti-cancer agent or at least one other anti-cancer agent.

In some embodiments, the amount of the compound and the amount of the anti-cancer agent are each periodically administered to the subject

In some embodiments, the amount of the compound and the amount of the anti-cancer agent are administered simultaneously, separately or sequentially.

In some embodiments, the method comprising administering to the subject an effective amount of the compound and subsequently administering to the subject, after an interval comprising at least 1 hour, the anti-cancer agent.

In some embodiments, the method wherein the compound inhibits protein phosphatase 2A (PP2A) in the subject.

In some embodiments, the method wherein the compound inhibits one or more cellular pathways that repair cellular damage of the ovarian cancer cells which is caused by the anti-cancer agent.

In some embodiments, the method wherein the compound induces hyperphosphorylation of Chk1, BRCA1, Wee1, and/or γH2AX in the subject.

In some embodiments, the method wherein the compound increases abrogation of G2/M arrest of ovarian cancer cells.

In some embodiments, the method wherein the amount of compound administered is 0.025-0.25 mg/kg/day.

In some embodiments, the method wherein the amount of compound administered is 0.05-0.25 mg/kg/day.

In some embodiments, the method wherein the amount of compound administered is 0.1-0.15 mg/kg/day.

In some embodiments, the method wherein the amount of compound administered is 0.2-0.25 mg/kg/day.

In some embodiments, the method wherein the amount of compound administered is 2.5-15 mg/day.

In some embodiments, the method wherein the amount of compound administered is 5.0-15 mg/day.

In some embodiments, the method wherein the amount of compound administered is 7.5-15 mg/day.

In some embodiments, the method wherein the amount of compound administered is 7.5-12.5 mg/day.

In some embodiments, the method wherein the amount of compound administered is 10-15 mg/day.

In some embodiments, the method wherein the amount of anti-cancer agent administered is 0.05-0.3 mg/kg/day.

In some embodiments, the method wherein the amount of anti-cancer agent administered is 0.1-0.3 mg/kg/day.

In some embodiments, the method wherein the amount of anti-cancer agent administered is 0.1-0.15 mg/kg/day.

In some embodiments, the method wherein the amount of anti-cancer agent administered is 0.225-0.275 mg/kg/day.

In some embodiments, the method wherein the amount of anti-cancer agent administered is 2.5-20 mg/day.

In some embodiments, the method wherein the amount of anti-cancer agent administered is 5-20 mg/day.

In some embodiments, the method wherein the amount of anti-cancer agent administered is 5-10 mg/day.

In some embodiments, the method wherein the amount of anti-cancer agent administered is 12.5-17.5 mg/day.

In some embodiments, the anti-cancer agent is a platinum-based anti-cancer agent.

In some embodiments, the platinum-based anti-cancer agent is cisplatin, carboplatin, oxaliplatin, satraplatin, picoplatin, nedaplatin, triplatin or lipoplatin.

In some embodiments, the platinum-based anti-cancer agent is cisplatin.

In some embodiments, the anti-cancer agent is an anthracycline anti-cancer agent.

In some embodiments, the anthracycline anti-cancer agent is doxorubicin, daunorubicin, epirubicin, idarubicin, or valrubicin.

In some embodiments, the anthracycline anti-cancer agent is doxorubicin.

In some embodiments, the ovarian cancer is refractory.

In some embodiments, the subject is a human.

In some embodiments, the human subject was previously treated with the anti-cancer agent and the ovarian cancer developed resistance to the anti-cancer agent.

In some embodiments of the method, the compound has the structure:

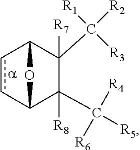

In some embodiments of the method, bond α in the compound is present.

In some embodiments of the method, bond α in the compound is absent.

In some embodiments of the method, the compound wherein
R₃ is OH, O⁻, or OR₉,
   wherein R₉ is alkyl, alkenyl, alkynyl or aryl;
R₄ is

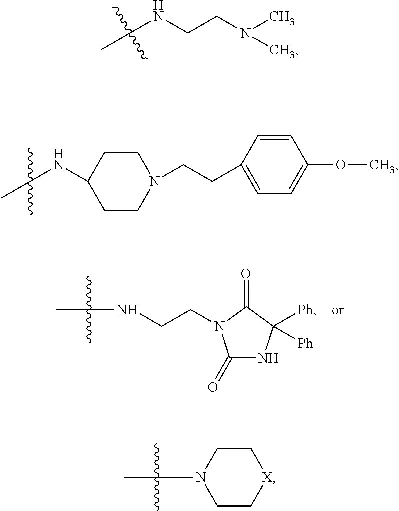

where X is O, S, NR₁₀, N⁺HR₁₀ or N⁺R₁₀R₁₀,
   where each R₁₀ is independently H, alkyl, alkenyl, alkynyl, aryl,

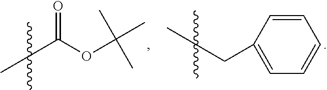

In some embodiments of the method, the compound wherein
R₃ is OH, O⁻ or OR₉,
   where R₉ is H, methyl, ethyl or phenyl.

In some embodiments of the method, the compound wherein
R₃ is OH, O⁻ or OR₉,
   wherein R₉ is methyl.

In some embodiments of the method, the compound wherein
R₄ is

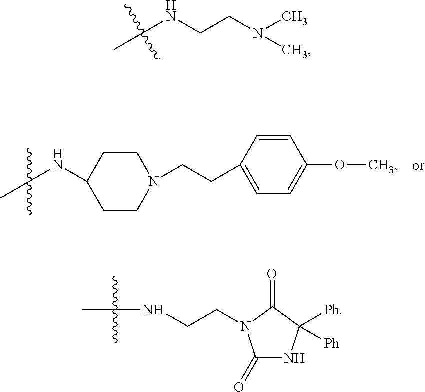

In some embodiments of the method, the compound wherein
R₄ is

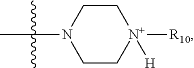

wherein R₁₀ is H, alkyl, alkenyl, alkynyl, aryl, or

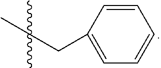 [SMILES: *Cc1ccccc1]

In some embodiments of the method, the compound wherein
R₄ is

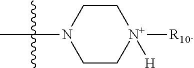

wherein R₁₀ is —H, —CH₃, —CH₂CH₃, or

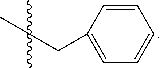 [SMILES: *Cc1ccccc1]

In some embodiments of the method, the compound wherein
R₄ is

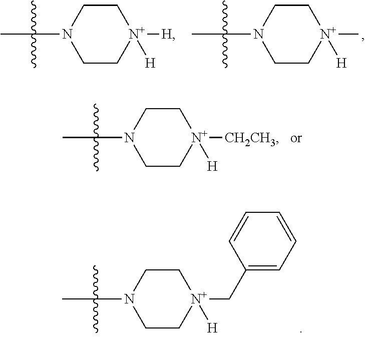

In some embodiments of the method, the compound wherein
R₄ is

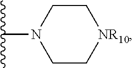

wherein R₁₀ is H, alkyl, alkenyl, alkynyl, aryl,

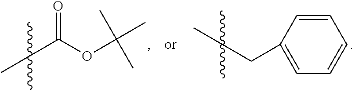

In some embodiments of the method, the compound wherein
R₄ is

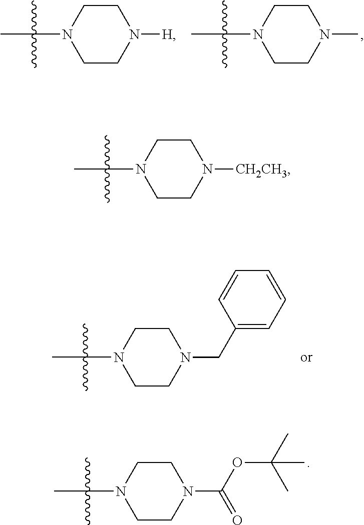

In some embodiments of the method, the compound wherein
R₄ is

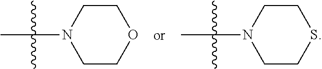 [SMILES: *N1CCOCC1 ; *N1CCSCC1]

In some embodiments of the method, the compound has the structure:

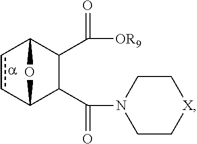

wherein
bond α is present or absent;
R₉ is present or absent and when present is H, alkyl, alkenyl, alkynyl or phenyl; and
X is O, NR₁₀, NH⁺R₁₀ or N⁺R₁₀R₁₀,
   where each R₁₀ is independently H, alkyl, substituted alkyl, alkenyl, substituted alkenyl, alkynyl, substituted alkynyl, aryl,

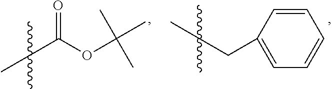

—CH₂CN, —CH₂CO₂R₁₂, or —CH₂COR₁₂,
   where R₁₂ is H or alkyl,
      or a salt, zwitterion or ester thereof.

In some embodiments of the method, the compound has the structure:

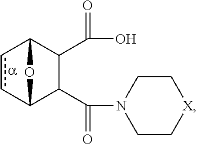

wherein
bond α is present or absent;
X is O or NR₁₀,
   where each R₁₀ is independently H, alkyl, substituted alkyl, alkenyl, substituted alkenyl, alkynyl, substituted alkynyl, aryl,

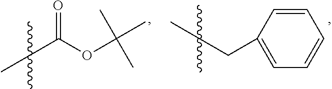

—CH₂CN, —CH₂CO₂R₁₂, or —CH₂COR₁₂,
   where R₁₂ is H or alkyl,
      or a salt, zwitterion or ester thereof.

In some embodiments of the method, the compound has the structure:

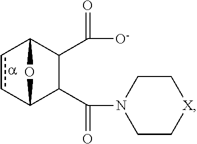

wherein
bond α is present or absent;
X is O or NH⁺R₁₀,
   where R₁₀ is H, alkyl, substituted alkyl, alkenyl, substituted alkenyl, alkynyl, substituted alkynyl, aryl,

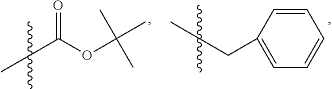

—CH₂CN, —CH₂CO₂R₁₂, or —CH₂COR₁₂,
   where R₁₂ is H or alkyl,
      or a salt, zwitterion or ester thereof.

In some embodiments of the method, the compound has the structure:

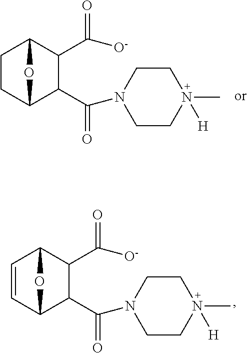

or a salt or ester thereof.

In one embodiment, the compound of the method has the structure:

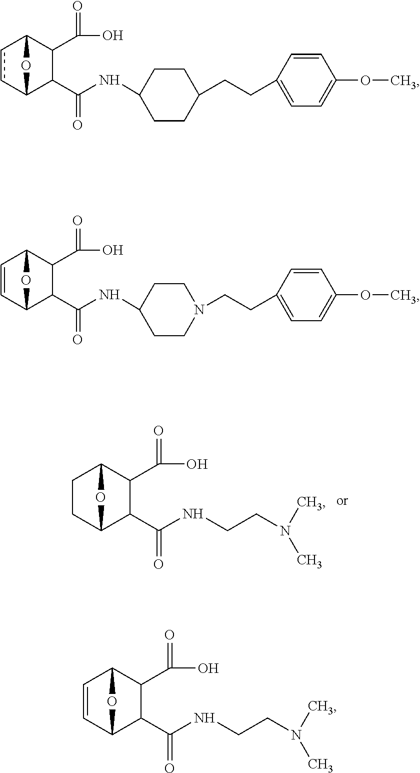

or a salt, zwitterion, or ester thereof.

In one embodiment, the compound of the method has the structure:

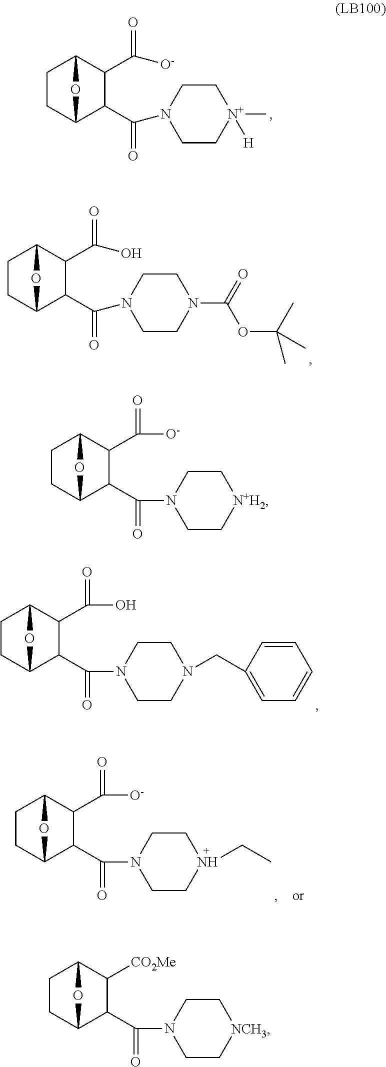

or a salt, zwitterion, or ester thereof.

In one embodiment, the compound of the method has the structure:

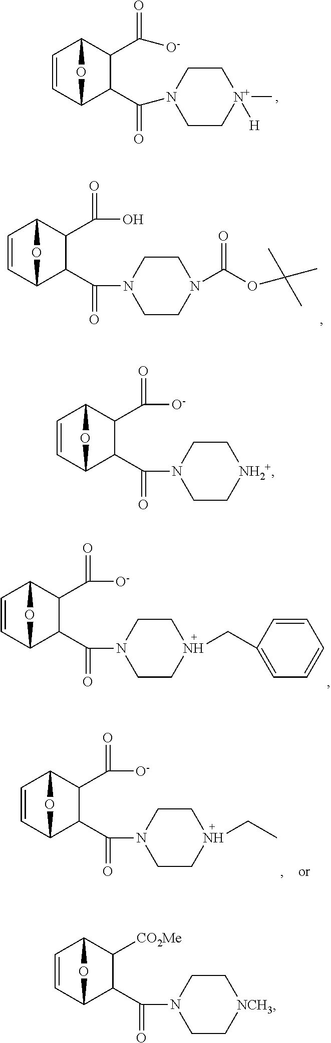

or a salt, zwitterion, or ester thereof.

In one embodiment, the compound of the method has the structure:

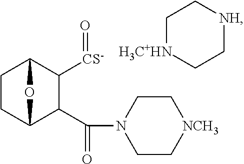

or a salt, zwitterion, or ester thereof.

The present invention also provides a method of treating ovarian cancer in a subject afflicted therewith comprising administering to the subject an effective amount of an anti-cancer agent and an effective amount of a compound having the structure:

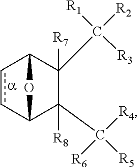

wherein
bond α is present or absent;
R₁ and R₂ together are ═O;
R₃ and R₄ are each different, and each is O(CH₂)₁₋₆R₉ or OR₉, or

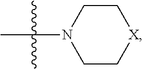

where X is O, S, NR₁₀, N⁺HR₁₀ or N⁺R₁₀R₁₀,
   where each R₉ is H, alkyl, C₂-C₁₂ alkyl substituted alkyl, alkenyl, alkynyl, aryl, (C₆H₅)(CH₂)₁₋₆(CHNHBOC)CO₂H, (C₆H₅)(CH₂)₁₋₆(CHNH₂)CO₂H, (CH₂)₁₋₆(CHNHBOC)CO₂H, (CH₂)₁₋₆(CHNH₂)CO₂H or (CH₂)₁₋₆CCl₃,
   where each R₁₀ is independently H, alkyl, hydroxyalkyl, C₂-C₁₂ alkyl, alkenyl, C₄-C₁₂ alkenyl, alkynyl, aryl,

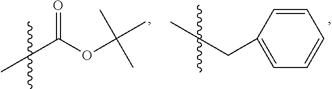

—CH₂CN, —CH₂CO₂R₁₁, or —CH₂COR₁₁,
   where each R₁₁ is independently alkyl, alkenyl or alkynyl, each of which is substituted or unsubstituted, or H;
or R₃ and R₄ are each different and each is OH or

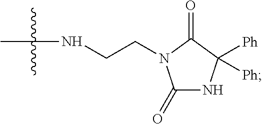 [SMILES: *NCCN1C(=O)NC(c2ccccc2)(c2ccccc2)C1=O]

R₅ and R₆ taken together are ═O;
R₇ and R₈ are each H; and
each occurrence of alkyl, alkenyl, or alkynyl is branched or unbranched, unsubstituted or substituted,
   or a salt, zwitterion, or ester thereof,
   so as to thereby treat the ovarian cancer in the subject.

In one embodiment, the compound of the method has the structure:

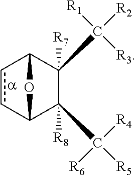

In one embodiment of the method, the bond α is present.

In one embodiment of the method, the bond α is absent.

In one embodiment of the method,
R₃ is OR₉ or O(CH₂)₁₋₆R₉,
   where R₉ is aryl, substituted ethyl or substituted phenyl,
   wherein the substituent is in the para position of the phenyl;
R₄ is

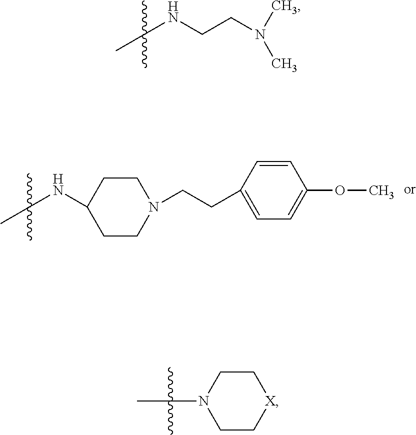

where X is O, S, NR₁₀, or N⁺R₁₀R₁₀,
where each R₁₀ is independently H, alkyl, hydroxyalkyl, substituted C₂-C₁₂ alkyl, alkenyl, substituted C₄-C₁₂ alkenyl, alkynyl, substituted alkynyl, aryl,

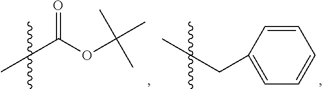

—CH₂CN, —CH₂CO₂R₁₁, —CH₂COR₁₁,
   where R₁₁ is alkyl, alkenyl or alkynyl, each of which is substituted or unsubstituted, or H;
      or where R₃ is OH and R₄ is

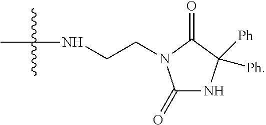 [SMILES: *NCCN1C(=O)NC(c2ccccc2)(c2ccccc2)C1=O]

In one embodiment of the method,

### R₄ is

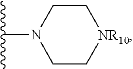 [0223] where R₁₀ is alkyl or hydroxylalkyl.

In one embodiment of the method,
R₁ and R₂ together are ═O;
R₃ is OR₉ or O(CH₂)₁₋₂R₉,
   where R₉ is aryl, substituted ethyl, or substituted phenyl,
   wherein the substituent is in the para position of the phenyl;
R₄ is

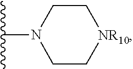

where R₁₀ is alkyl or hydroxyl alkyl;
R₅ and R₆ together are ═O; and
R₇ and R₈ are each independently H.

In one embodiment of the method,
R₁ and R₂ together are ═O;
R₃ is O(CH₂)R₉, or OR₉,
   where R₉ is phenyl or CH₂CCl₃,

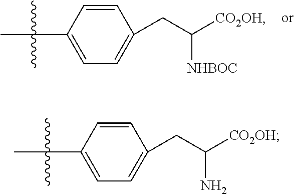

R₄ is

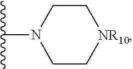

where R₁₀ is CH₃ or CH₃CH₂OH;
R₅ and R₆ together are ═O; and
R₇ and R₈ are each independently H.

In one embodiment of the method,
R₃ is OR₉,
   where R₉ is (CH₂)₁₋₆ (CHNHBOC)CO₂H, (CH₂)₁₋₆ (CHNH₂)CO₂H, or (CH₂)₁₋₆CCl₃.

In one embodiment of the method,
R₉ is CH₂ (CHNHBOC)CO₂H, CH₂ (CHNH₂)CO₂H, or CH₂CCl₃.

In one embodiment of the method,
R₉ is (C₆H₅)(CH₂)₁₋₆(CHNHBOC)CO₂H or (C₆H₅)(CH₂)₁₋₆ (CHNH₂)CO₂H.

In one embodiment of the method,
R₉ is (C₆H₅)(CH₂)(CHNHBOC)CO₂H or (C₆H₅)(CH₂)(CHNH₂)CO₂H

In one embodiment of the method,
R₃ is O(CH₂)₂₋₆R₉ or O(CH₂)R₉,
   where R₉ is phenyl.

In one embodiment of the method,
R₃ is OH and R₄ is

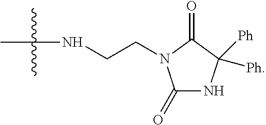 [SMILES: *NCCN1C(=O)NC(c2ccccc2)(c2ccccc2)C1=O]

In one embodiment of the method,
R₄ is

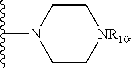

wherein R₁₀ is alkyl or hydroxyalkyl.

In one embodiment of the method, R₁₁ is —CH₂CH₂OH or —CH₃.

In one embodiment of the method, the compound has the structure:

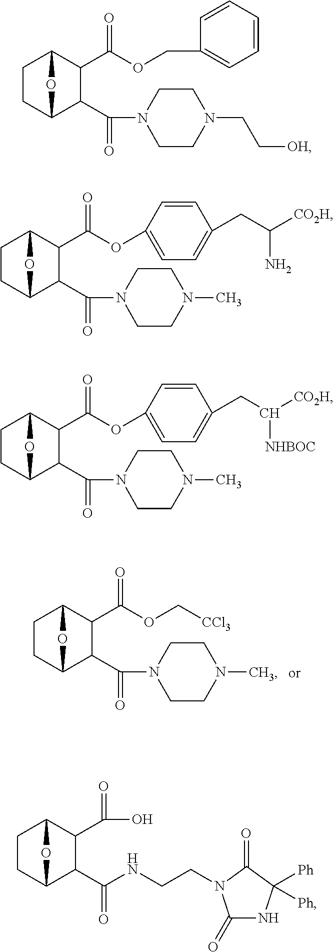

or a salt, zwitterion, or ester thereof.

In one embodiment of the method, the compound has the structure:

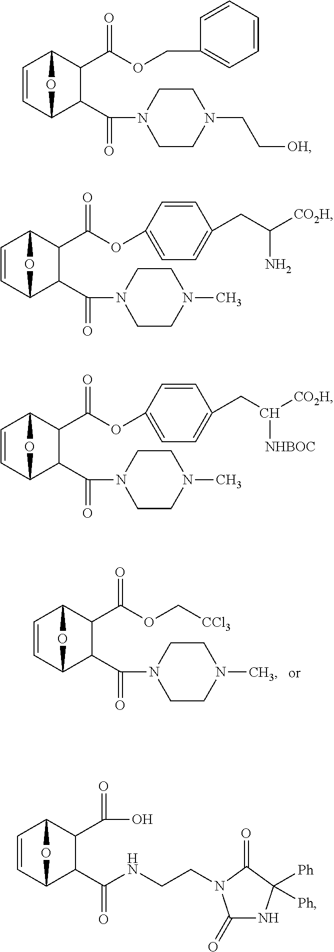

or a salt, zwitterion, or ester thereof.

The present invention also provides a method of treating ovarian cancer in a subject afflicted therewith comprising administering to the subject an effective amount of an anti-cancer agent and an effective amount of a compound having the structure:

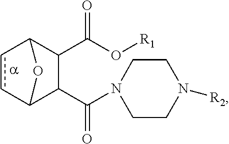

wherein
bond α is absent or present;
R₁ is C₂-C₂₀ alkyl, C₂-C₂₀ alkenyl, or C₂-C₂₀ alkynyl;
R₂ is H, C₁-C₁₂ alkyl, C₁-C₁₂ alkenyl, C₁-C₁₂ alkynyl, C₁-C₁₂ alkyl-(phenyl), C₁-C₂₂ alkyl-(OH), or C(O)C(CH₃)₃,
   or a salt, zwitterion, or ester thereof,
   so as to thereby treat the ovarian cancer in the subject.

In some embodiments, the compound has the structure:

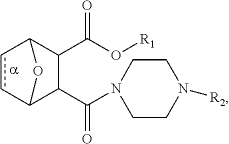

wherein
bond α is absent or present;
R₁ is C₃-C₂₀ alkyl, C₂-C₂₀ alkenyl, or C₂-C₂₀ alkynyl;
R₂ is H, C₁-C₁₂ alkyl, C₁-C₁₂ alkenyl, C₁-C₁₂ alkynyl, C₁-C₁₂ alkyl-(phenyl), C₁-C₁₂ alkyl-(OH), or C(O)C(CH₃)₃,
   or a salt, zwitterion, or ester thereof.

In some embodiments, the compound has the structure:

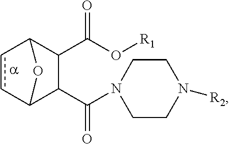

wherein
bond α is absent or present;
R₁ is C₄-C₂₀ alkyl, C₂-C₂₀ alkenyl, or C₂-C₂₀ alkynyl;
R₂ is H, C₁-C₁₂ alkyl, C₁-C₁₂ alkenyl, C₁-C₁₂ alkynyl, C₁-C₁₂ alkyl-(phenyl), C₁-C₁₂ alkyl-(OH), or C(O)C(CH₃)₃,
   or a salt, zwitterion, or ester thereof.

In some embodiments, the above compound having the structure:

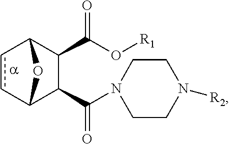

or a salt, zwitterion, or ester thereof.

In some embodiments, the above compound wherein
R₁ is —CH₂CH₃,
—CH₂CH₂CH₃,
—CH₂CH₂CH₂CH₃,
—CH₂CH₂CH₂CH₂CH₃,
—CH₂CH₂CH₂CH₂CH₂CH₃,
—CH₂CH₂CH₂CH₂CH₂CH₂CH₃,
—CH₂CH₂CH₂CH₂CH₂CH₂CH₂CH₃,
—CH₂CH₂CH₂CH₂CH₂CH₂CH₂CH₂CH₃, or
—CH₂CH₂CH₂CH₂CH₂CH₂CH₂CH₂CH₂CH₃.

In some embodiments, the above PP2A inhibitor wherein R₁ is —CH₂CH₂CH₂CH₂CH₂CH₂CH₂CH₂CH₂CH₂CH₂CH₂CH₂CH₂CH₂CH₂CH₃, or —CH₂CH₂CH₂CH₂CH₂CH₂CH₂CH₂CH═CHCH₂CH═CHCH₂CH₂CH₂CH₂CH₃.

In some embodiments, the above compound wherein
R₂ is —H, —CH₃, —CH₂CH₃, —CH₂-phenyl, —CH₂CH₂—OH, or —C(O)C(CH₃)₃.

In some embodiments, the compound having the structure:

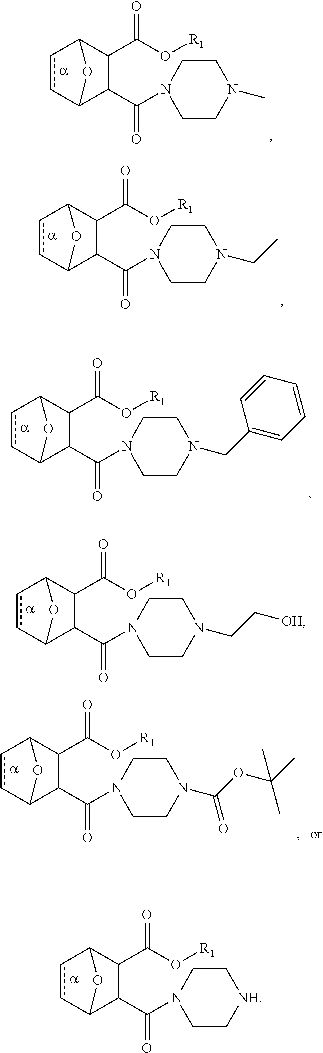

In some embodiments, the above compound wherein α is absent.

In some embodiments, the above compound wherein α is present.

In some embodiments, the compound having the structure:

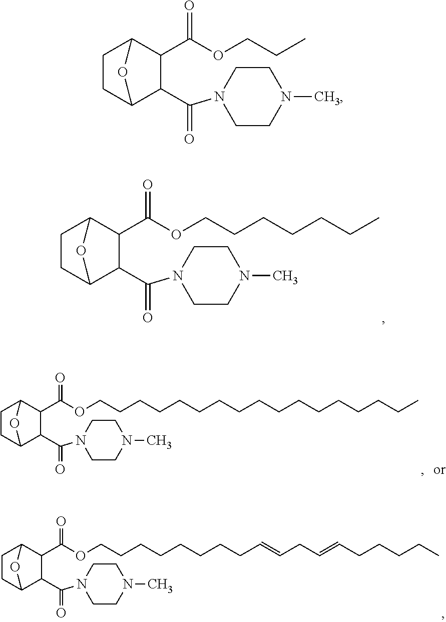 [SMILES: CCCOC(=O)C1C2CCC(O2)C1C(=O)N1CCN(C)CC1 ; CCCCCCCOC(=O)C1C2CCC(O2)C1C(=O)N1CCN(C)CC1 ; CCCCCCCCCCCCCCCCCOC(=O)C1C2CCC(O2)C1C(=O)N1CCN(C)CC1 ; CCCCC/C=C/C/C=C/CCCCCCCCOC(=O)C1C2CCC(O2)C1C(=O)N1CCN(C)CC1]

or a salt, zwitterion, or ester thereof.

The analogs of LB-100 disclosed herein have analogous activity to LB-100 and behave similarly in the assays disclosed herein.

The present invention provides a pharmaceutical composition comprising a compound of the present invention and an anticancer agent, and at least one pharmaceutically acceptable carrier for use in treating ovarian cancer.

In some embodiments, the pharmaceutical composition wherein the pharmaceutically acceptable carrier comprises a liposome.

In some embodiments, the pharmaceutical composition wherein the compound is contained in a liposome or microsphere, or the compound and the anti-cancer agent are contained in a liposome or microsphere.

The present invention provides a pharmaceutical composition comprising an amount of the compound of the present invention for use in treating a subject afflicted with ovarian cancer as an add-on therapy or in combination with, or simultaneously, contemporaneously or concomitantly with an anti-cancer agent.

In some embodiments, the compound of the present invention for use as an add-on therapy or in combination with an anti-cancer agent in treating a subject afflicted with ovarian cancer.

In some embodiments, the compound of the present invention in combination with an anti-cancer agent for use in treating ovarian cancer.

In some embodiments, a product containing an amount of the compound of the present invention and an amount of an anti-cancer agent for simultaneous, separate or sequential use in treating a subject afflicted ovarian cancer.

In some embodiments of any of the above methods or uses, the subject is a human.

In some embodiments of any of the above methods or uses, the compound and/or anti-cancer agent is orally administered to the subject.

For the foregoing embodiments, each embodiment disclosed herein is contemplated as being applicable to each of the other disclosed embodiments. Thus, all combinations of the various elements described herein are within the scope of the invention.

The compounds used in the method of the present invention are protein phosphatase 2A (PP2A) inhibitors. Methods of preparation may be found in Lu et al., 2009; U.S. Pat. No. 7,998,957 B2; and U.S. Pat. No. 8,426,444 B2. Compound LB-100 is an inhibitor of PP2A in vitro in human cancer cells and in xenografts of human tumor cells in mice when given parenterally in mice. LB-100 inhibits the growth of cancer cells in mouse model systems.

In some embodiments, the ovarian cancer is advanced or has metastasized in patients whose disease has not gotten better with other types of treatment or chemotherapy.

In some embodiments, the cancer is drug resistant ovarian cancer. In some embodiments, the ovarian cancer is advanced ovarian cancer. In some embodiments, the ovarian cancer is unrespectable ovarian cancer. In some embodiments, the ovarian cancer is stage I, II, II or IV ovarian cancer.

In some embodiments, the ovarian cancer is advanced and/or cannot be treated with surgery or radiation therapy.

In some embodiments, the subject afflicted with ovarian cancer has already had surgery or radiation therapy.

In some embodiments, the ovarian cancer was previously treated with an anti-cancer agent.

In some embodiments, the ovarian cancer was previously treated with cisplatin.

In one embodiment of any of the above methods, the method consisting essentially of administering the compound and the anti-cancer agent.

In some embodiments, the ovarian cancer has developed resistance to at least one drug. For example, a drug resistant cancer may have developed drug-resistance to vinca alkaloids (e.g., vinblastine, vincristine, and vinorelvine); anthracyclines (e.g., doxorubicin, daunorubicin, and idarubicin); microtubule-stabilizing drug paclitaxel; drugs that target tyrosine kinases (TKs) activity (e.g., dasatinib, nilotinib, and imatinib); or platinum-based antineoplastic drugs (e.g., cisplatin).

As used herein, a “symptom” associated with reperfusion injury includes any clinical or laboratory manifestation associated with reperfusion injury and is not limited to what the subject can feel or observe.

As used herein, “treatment of the diseases” or “treating”, e.g. of reperfusion injury, encompasses inducing prevention, inhibition, regression, or stasis of the disease or a symptom or condition associated with the disease.

As used herein, “inhibition” of disease progression or disease complication in a subject means preventing or reducing the disease progression and/or disease complication in the subject.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2, . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₂₀ alkyl, C₂-C₂₀ alkyl, C₃-C₂₀ alkyl, C₄-C₂₀ alkyl and so on. An embodiment can be C₁-C₃₀ alkyl, C₂-C₃₀ alkyl, C₃-C₃₀ alkyl, C₄-C₃₀ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2, . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl, C₃-C₁₂ alkenyl, C₂-C₂₀ alkenyl, C₃-C₂₀ alkenyl, C₂-C₃₀ alkenyl, or C₃-C₃₀ alkenyl.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2, . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups, include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-C₁₂ alkynyl, C₂-C₂₀ alkynyl, C₃-C₂₀ alkynyl, C₂-C₃₀ alkynyl, or C₃-C₃₀ alkynyl.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. The substituted aryls included in this invention include substitution at any suitable position with amines, substituted amines, alkylamines, hydroxys and alkylhydroxys, wherein the “alkyl” portion of the alkylamines and alkylhydroxys is a C₂-Cₙ alkyl as defined hereinabove. The substituted amines may be substituted with alkyl, alkenyl, alkynl, or aryl groups as hereinabove defined.

Each occurrence of alkyl, alkenyl, or alkynyl is branched or unbranched, unsubstituted or substituted.

The alkyl, alkenyl, alkynyl, and aryl substituents may be unsubstituted or unsubstituted, unless specifically defined otherwise. For example, a (C₁-C₆) alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, and alkynyl groups can be further substituted by replacing one or more hydrogen atoms by non-hydrogen groups described herein to the extent possible. These include, but are not limited to, halo, hydroxy, mercapto, amino, carboxy, cyano and carbamoyl.

The term “substituted” as used herein means that a given structure has a substituent which can be an alkyl, alkenyl, or aryl group as defined above. The term shall be deemed to include multiple degrees of substitution by a named substitutent. Where multiple substituent moieties are disclosed or claimed, the substituted compound can be independently substituted by one or more of the disclosed or claimed substituent moieties, singly or plurality. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The following delivery systems, which employ a number of routinely used pharmaceutical carriers, may be used but are only representative of the many possible systems envisioned for administering compositions in accordance with the invention.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's).

Other injectable drug delivery systems include solutions, suspensions, gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

The compounds used in the method of the present invention may be in a salt form. As used herein, a “salt” is a salt of the instant compounds which has been modified by making acid or base salts of the compounds. In the case of compounds used to treat an infection or disease, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The present invention includes esters or pharmaceutically acceptable esters of the compounds of the present method. The term “ester” includes, but is not limited to, a compound containing the R—CO—OR′ group. The “R—CO—O” portion may be derived from the parent compound of the present invention. The “R′” portion includes, but is not limited to, alkyl, alkenyl, alkynyl, heteroalkyl, aryl, and carboxy alkyl groups.

The present invention includes pharmaceutically acceptable prodrug esters of the compounds of the present method. Pharmaceutically acceptable prodrug esters of the compounds of the present invention are ester derivatives which are convertible by solvolysis or under physiological conditions to the free carboxylic acids of the parent compound. An example of a pro-drug is an alkly ester which is cleaved in vivo to yield the compound of interest.

The compound, or salt, zwitterion, or ester thereof, is optionally provided in a pharmaceutically acceptable composition including the appropriate pharmaceutically acceptable carriers.

As used herein, an “amount” or “dose” of an agent measured in milligrams refers to the milligrams of agent present in a drug product, regardless of the form of the drug product.

The National Institutes of Health (NIH) provides a table of Equivalent Surface Area Dosage Conversion Factors below (Table A) which provides conversion factors that account for surface area to weight ratios between species.

TABLE A
Equivalent Surface Area Dosage Conversion Factors

| | | To | | | | |
|---|---|---|---|---|---|---|
| | | Mouse | Rat | Monkey | Dog | Man |
| | | 20 g | 150 g | 3 kg | 8 kg | 60 kg |
| From | Mouse | 1 | ½ | ¼ | 1/6 | 1/12 |
| | Rat | 2 | 1 | ½ | ¼ | 1/7 |
| | Monkey | 4 | 2 | 1 | ⅗ | ⅓ |
| | Dog | 6 | 4 | 1⅔ | 1 | ½ |
| | Man | 12 | 7 | 3 | 2 | 1 |

As used herein, the term “therapeutically effective amount” or “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

Where a range is given in the specification it is understood that the range includes all integers and 0.1 units within that range, and any sub-range thereof. For example, a range of 77 to 90% is a disclosure of 77, 78, 79, 80, and 81% etc.

As used herein, “about” with regard to a stated number encompasses a range of +one percent to −one percent of the stated value. By way of example, about 100 mg/kg therefore includes 99, 99.1, 99.2, 99.3, 99.4, 99.5, 99.6, 99.7, 99.8, 99.9, 100, 100.1, 100.2, 100.3, 100.4, 100.5, 100.6, 100.7, 100.8, 100.9 and 101 mg/kg. Accordingly, about 100 mg/kg includes, in an embodiment, 100 mg/kg.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

All combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Material and Methods

### Cell Lines, Cell Culture, and Drug Solutions

SKOV-3 ovarian cancer cells were purchased from American Type Culture Collection (ATCC) (Manassas, Va.). SKOV-3 cells were cultured in McCoy's 5A medium (ATCC, Manassas, Va.) supplemented with 10% fetal bovine serum and 100 units/mL penicillin G sodium, 100 ug/mL streptomycin sulfate, and 292 μg/ml, L-glutamine (BioWhittaker, Wakersville, Md.). Luciferase-expressing cells were generated by infecting SKOV-3 cells with pCLNCX-luciferase retrovirus (SKOV-3-Luc) as previously reported (Wei, B. R. et al. 2009). Human OVCAR-8 ovarian cancer cells were provided by the National Cancer Institute (part of the NCI-60 collection). The PEO1, PEO4, and PEO6 ovarian cancer cell lines have previously been characterized (Langdon, S. P. et al. 1988) and were kindly provided by Dr. Ian Goldlust (National Center for Advancing Translational Sciences, Shady Grove, Md.). All the PEO cells and OVCAR-8 cells were cultured in RPMI medium (Invitrogen, Carlsbad, Calif.) supplemented with 10% fetal bovine serum and 100 units/mL penicillin G sodium, 100 μg/mL streptomycin sulfate, and 292 μg/mL L-glutamine (BioWhittaker). Cisplatin was purchased from Sigma-Aldrich (St Louis, Mo.) and dissolved in sterile saline solution (0.9%), prior to administration. It was recently shown that the cytotoxic efficacy of cisplatin is significantly lost when dissolved in DMSO compared to saline/PBS (Hall, M. D. et al. 2014); for this reason, normal saline was used as the solvent. LB100 was diluted in sterile PBS prior to administration.

### PP2a Phosphatase Activity Assay

Ovarian cancer cells were grown to 80% confluence in 100 mm dishes and treated with LB100 as indicated and prepared as described previously (Wei, D. et al. 2013). Following treatment for 2 h, cells were washed twice with cold PBS (pH7.4) and lysed in lysis buffer (20 mmol/L imidazole-HCL, 2 mmol/L EDTA, 2 mmol/L EGTA, pH 7.0) supplemented with protease inhibitors (Roche) for 30 minutes on ice. Cell lysates were sonicated for 10 s then centrifuged at 2,000×g for 5 min. Supernatants were assayed with the PP2A Phosphatase Assay Kit (Millipore, Billerica, Mass.) according to the manufacturer's instructions. Experiments were performed in triplicate, and the data are presented as a percent mean of relative PP2A activity compared to control ±SD.

### MTT Assay

Cell survival was measured by the 3-(4,5-dimethylthiazol-2-yl)-2,5-diphenyltetrazolium bromide (MTT, Invitrogen) assay. Cells were seeded at a density of 5,000 cells per well in 96-well plates and incubated at 37° C. in humidified 5% CO₂ for 24 hours. The 50% inhibitory concentration (IC₅₀) values were defined as the drug concentrations required to reduce cellular proliferation to 50% of the untreated control well. For IC₅₀ determination, serially diluted LB100 or cisplatin was added to give the intended final concentrations. All MTT assays were carried out according to the manufacturer's instructions (Molecular Probes, Eugen, Oreg.). Absorbance values were determined at 570 nM on a Spectra Max 250 spectrophotometer (Molecular Devices, Sunnyvale, Calif.). All MTT assays were performed in triplicate. In order to determine if LB100 could enhance the cytotoxic effect of cisplatin, cells were pretreated with either a non-toxic or slightly toxic dose of LB100 for 1 h prior to the addition of either a low or high dose of cisplatin. Cells were treated with both drugs for 72 h. Cell viability was analyzed via the MTT assay as described above. Experiments were performed in triplicate, and the data are presented as a percent mean±SD.

Production of stable NT-shRNA and PP2AC-shRNA expressing SKOV-3 and OVCAR-8 Cells

To stably knockdown expression of PP2AC, a pLKO.1-puro plasmid-based 0.30 shRNA targeting the sequence: TGGAACTTGACGATACTCTAA (clone ID:TRCN0000002483, Sigma-Aldrich) was employed (PP2AC-shRNA). Additionally, a non-targeting shRNA plasmid (NT-shRNA) that targets no known human sequence was utilized as a control. A primer containing the target sequence (CTGGTTACGAAGCGAATCCTT) along with a stem loop followed by the reverse target sequence was annealed to a complimentary primer and inserted into the EcoRI and AgeI sites of the pLKO.1-puro plasmid (Addgene number 10878). Lentiviral particles were produced via Lipofectamine 2000 (Invitrogen)-mediated triple transfection of 293T cells with either the PP2AC-shRNA or the NT-shRNA along with the lentiviral envelope plasmid (pMD2.G, Addgene number 12259) and the lentiviral packaging plasmid (psPAX2, Addgene number 12260). Target cells (SKOV-3 and OVCAR-8 human ovarian cancer cell lines) were transduced with either PP2AC-shRNA or NT-shRNA containing lentiviral particles in the presence of [8 μg/mL]polybrene and stable cells were selected using [2 μg/mL] puromycin.

### Cell-Cycle Analysis

SK-OV-3 and OVCAR 8 cells were incubated in 100 mm³ sterile petri dishes for 24 h and treated with LB100, cisplatin, or LB100 plus cisplatin at indicated concentrations for 24 and 48 h. For cell-cycle analysis, cells were washed with PBS and fixed overnight in ice-cold 70% ethanol and stored at 4° C. Cells were then centrifuged and resuspended in 100 U RNAse (Sigma-Aldrich), and incubated at 37° C. for 20 min. Propidiumidodide solution (Invitrogen, 500 μL, 50 μg/mL in DPBS) was added to each tube and incubated in the dark at 4° C. overnight. Flow cytometry analysis was performed with CellQuestPro and data analysis was completed with ModFit LT. All data is in triplicate and presented as a percent mean±SD.

### Immunoblotting

Whole cell and homogenized tumor tissues were lysed in NP-40 lysis buffer [50 mM Tris/HCl, pH 7.4, 150 mM NaCl and 1% Nonidet P40, supplemented with Complete Protrase Inhibitor Cocktail tablets and PhosStop phosphatase inhibitors (Roche, Indianapolis, Ind.)] and prepared as previously described (Madigan, J. P. et al. 2009). Protein (40 μg) was resolved on SDS/PAGE (12% or 15% gels) and transferred onto Immobilon PVDF membrane. The membrane was then blocked for 1 hour at room temperature in 5% (w/v) non-fat milk in TBS-Tween and probed overnight with primary antibodies. After extensive washing, cells were probed with anti-rabbit or anti-mouse IgG-horseradish per oxidase (HRP)-conjugated secondary antibodies (Cell Signaling Technology, Danvers, Mass.) in blocking buffer for 1 h. Membranes were subsequently incubated in Immobilon Western blot Chemiluminescent HRP Substrate (Millipore) and developed on biomax XAR film (Kodak). Antibodies were purchased from Cell Signaling Technology: γH2AX (Ser139), p-Wee1 (Ser 642), Wee1, p-cdc2 (Tyr15), p-BRCA1 (Ser1524), p-Chk1 (Ser345), p-Chk1 (Ser317), Chk-1, phospho-Chk2 (Thr68), PP2Ac, cleaved caspase-3 (Asp175), cleaved PARP (Asp214), p-histone H3 (Ser10), p-ATR (Ser428), and p-(Ser)14-3-3 binding motif.

### In Vivo Intraperitoneal Ovarian Cancer Model

Five- to seven-week-old female nude athymic mice (nu/nu) were obtained from NCI (Frederick, Md.), maintained in accredited animal facilities and used as stipulated by the U.S. Public Health Service Policy on Humane Care and Use of Laboratory Animals, in accordance with institutional reviews (http://oacu.odnih.gov). 10⁶ SKOV-3/f-Luc cells were suspended in 100 μL PBS and injected into the intraperitoneal (i.p.) cavity. Tumor cells were allowed four days to become established, then the mice were randomized into four groups (4-5 animals per group): vehicle control (PBS), LB100 (1.5 mg/kg, i.p.), cisplatin (1.5 mg/kg, i.p.), and LB100 plus cisplatin (same doses as administered alone). Following tumor inoculation, mice were dosed on days 4, 6, 8, 10, 12 and 14. Dose and treatment schedule were established based on the activity of each agent reported in previous studies (Wei, D. et al. 2013; Lu, J. et al. 2009; Mabuchi, S. et al. 2007). For the combination group, LB100 was administered 1 h prior to cisplatin. Tumor growth was measured twice a week via bioluminescence imaging (BLI) as previously described (Bakhsheshian, J. et al. 2013). D-Luciferin (150 mg/kg, 3 mg/100 μL PBS) was administered via I.P. injection. Relative intensity of the BLI signal for each mouse was calculated by dividing the total luminescence for each session by the total luminescence measured on day 1 of treatment. Mice were continuously observed until indicated euthanasia endpoints (e.g. significant weight loss, ascites). Toxicity of the treatment regimens was assessed by the degree of weight loss and the overall health status was continuously monitored by a veterinarian on staff. For ex-vivo western blot analysis, four athymic female nude mice were treated with either saline (control), LB100 (1.5 mg/kg), cisplatin (2.5 mg/kg), or LB100 (1.5 mg/kg)+cisplatin (2.5 mg/kg). After 4 h, mice were euthanized and tumors were dissected from the intraperitoneal cavity, snap-frozen in liquid nitrogen, and lysed as described above.

### Statistical Analysis

Statistical analysis was performed using the software GraphPad Prism 6 (GraphPad Software, USA). Mean value was reported as mean±standard deviation, and a two-tailed unpaired t test was performed to assess statistical significance. Statistical significance was passed at two-sided p<0.05.

### Example 1. Ovarian Cancer Cell-Line Sensitivity to LS100 and Cisplatin

In order to characterize the effects of LB100 and cisplatin in ovarian carcinoma cells in vitro, six different cell lines carrying various p53 mutations were tested. SKOV-3 and OVCAR-8 cells have previously been described as p53 null and harboring an inactivating p53 mutation, respectively (Debernardis, D. et al. 1997). Both cell lines have also been characterized as intrinsically resistant to cisplatin (Kelland, L. R. et al. 1992; Kelland, L. R. et al. 1999; Taniguchi, T. et al. 2003). The PEO cell lines (PEO-1s, PEO-1m, PEO-4 and PEO-6) were generated from the same patient prior to (PEO-1s and PEO-1m) and following (PEO-4 and PEO-6) chemotherapy and acquired cisplatin resistance. The PEO-1 cell lines carry a BRCA2 missense (n) and STOP (s) mutation, respectively (Langdon, S. P. et al. 1988).

The 50% inhibitory concentration (IC₅₀) of each compound was determined using the MTT cytotoxicity assay (Table 1). Cell lines known to harbor intrinsic cisplatin resistance (SKOV-3, OVCAR-8) or acquired resistance (PEO-4, PEO-6) showed a 2- to 3-fold decreased sensitivity to cisplatin compared to PEO-1. SKOV-3 (IC₅₀=10.1±1.6 μM) was 2-fold less sensitive to LB100 compared to the other ovarian lines (average IC₅₀=5.7 μM), suggesting cell line-specific sensitivity to PP2A inhibition.

TABLE 1
Effect of cisplatin and LB100 on the viability
of ovarian cancer cell lines.

| Cell Line | LB100 (μM) | Cisplatin (μM) |
|---|---|---|
| SK-OV3 | 10.1 ± 1.8 | 7.6 ± 1.6 |
| OVCAR8 | 5.5 ± 0.5 | 7.2 ± 2.3 |
| PEO1-Missense | 6.2 ± 1.5 | 2.1 ± 0.4 |
| PEO1-STOP | 6.9 ± 1.0 | 2.3 ± 0.3 |
| PEO4 | 5.0 ± 0.6 | 4.3 ± 1.8 |
| PEO6 | 5.1 ± 0.2 | 8.0 ± 1.9 |

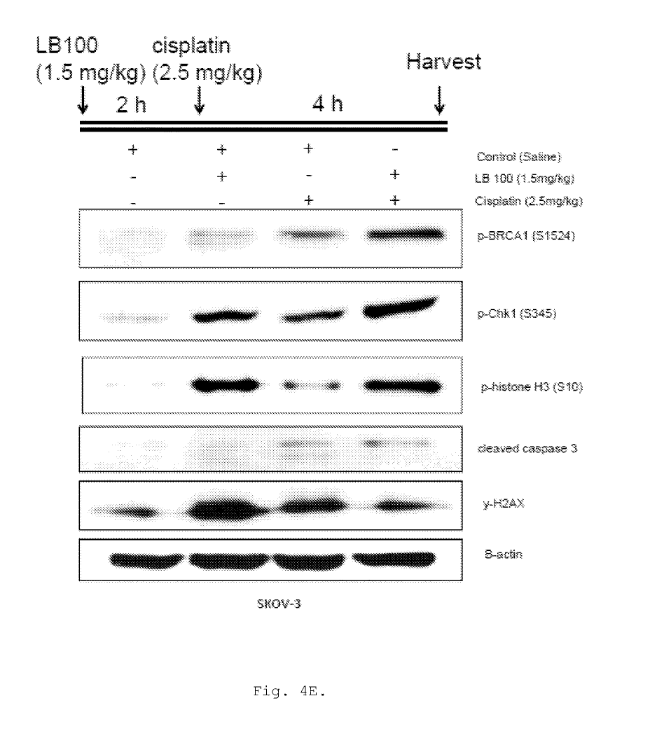

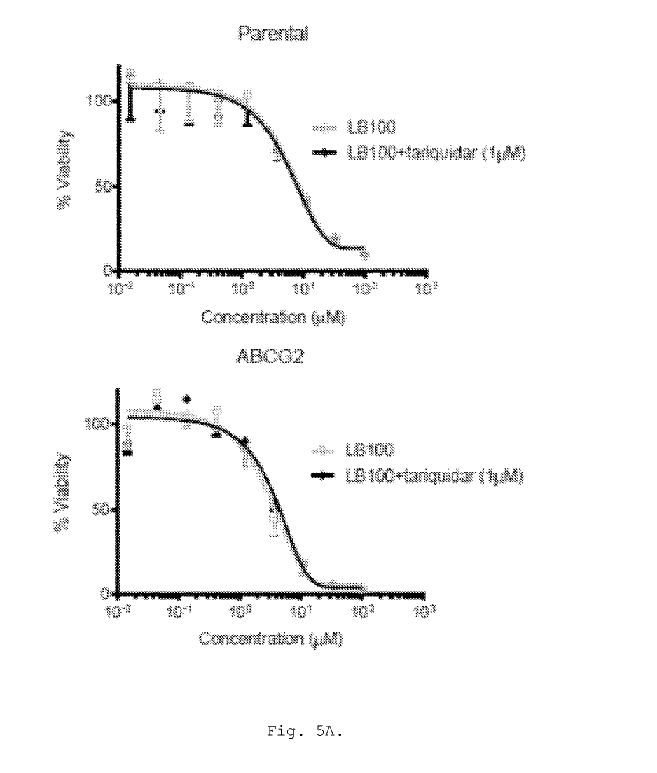

While ATP-binding cassette (ABC) efflux transporters have been shown to impact efficacy of Candidate small-molecule therapeutics (Kartner, N. et al. 1983), no information exists on whether this is the case for LB100. When HEK 293 human embryonic kidney cell lines overexpressing Pgp, MRP1, or ABCG2 (Robey, R. W. et al. 2005) were treated with equal concentration of LB100, the IC₅₀s of the transfected lines were not different compared with parent (non-transporter-expressing) cells or in the presence of an inhibitor (tariquidar) (FIG. 5). Cisplatin-resistant KB-CP.5 cells demonstrated two-fold increased resistance to LB100 compared with parental KB-3-1 human adenocarcinoma cells (FIG. 6).

### Example 2. LB100 Sensitizes Ovarian Cancer Cells to the Cytotoxic Effects of Cisplatin In-Vitro

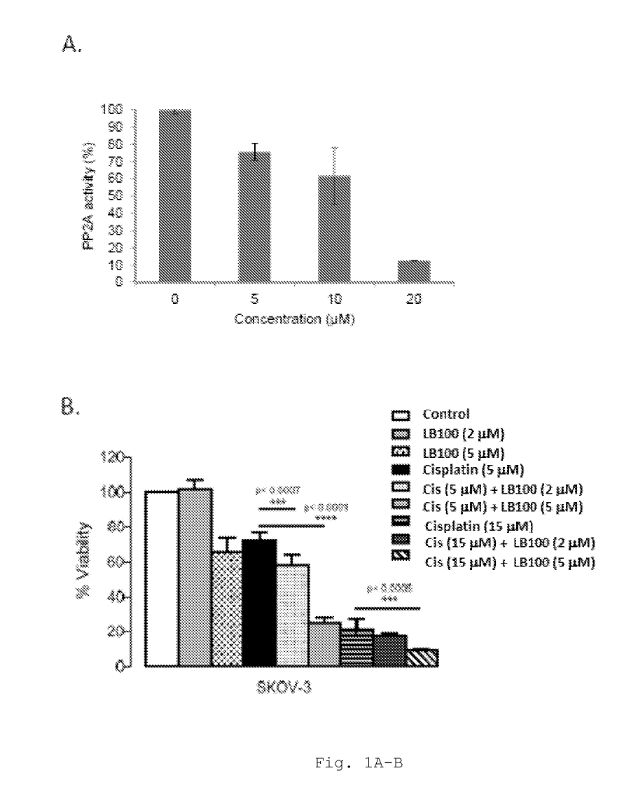

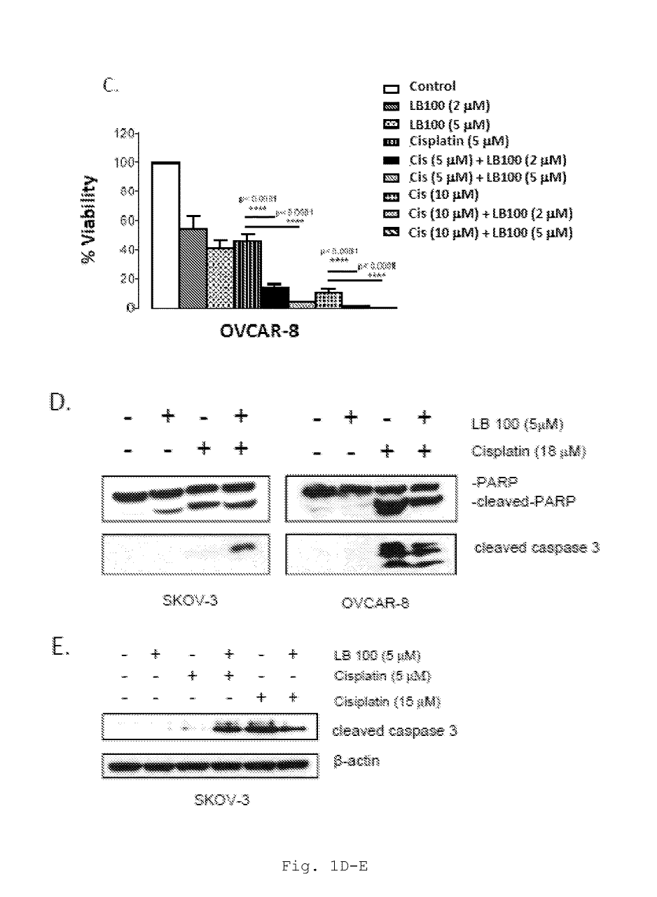

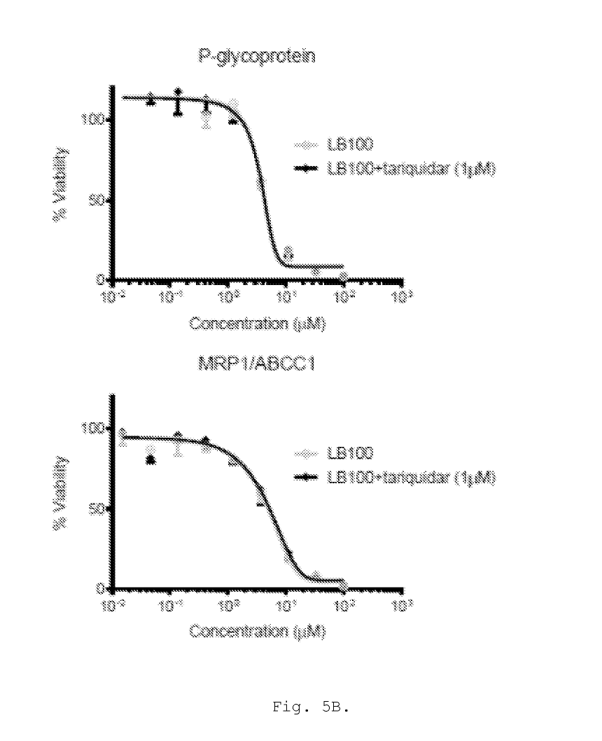

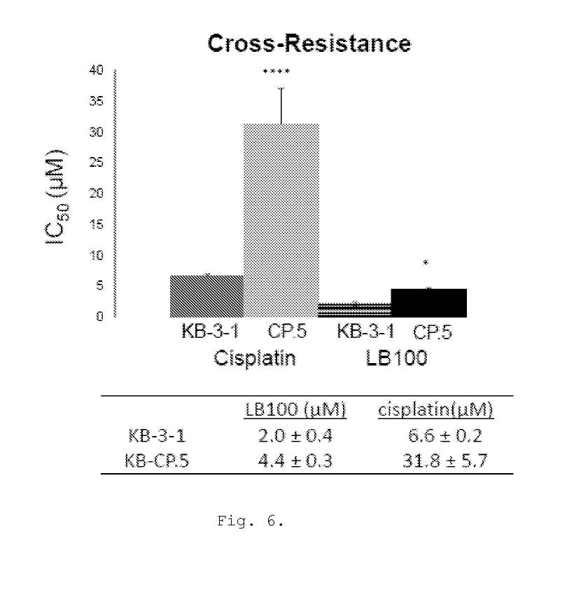

To determine whether PP2A inhibition with LB100 could sensitize ovarian cancer cells to the cytotoxic effects of cisplatin, the effect of LB100 on PP2A enzymatic activity was first assessed. Consistent with previous findings (Wei, D. et al. 2013; Lu, J. et al. 2009) LB100 alone caused a concentration-dependent decrease in PP2A enzymatic function in cell lysate from SKOV-3 cells (FIG. 1A). Next, cytotoxicity assays on ovarian cancer lines using either IC₂₅ or IC₇₅ doses of cisplatin in the presence of weakly-toxic doses (either less than IC₂₅ or less than IC₅₀) of LB100 (FIG. 1B, 1C, 7A, 7B, 7C,) were performed. Cells were pretreated with LB100 for 1 h prior to cisplatin. LB100 pre-treatment resulted in a significant decrease in cell viability compared to either treatment alone. For example, in SKOV-3 cells, 5 μM (IC₂₅) cisplatin alone resulted in 73±2% viability compared with control, but the presence of 2 μM and 5 μM LB100 significantly potentiated cell killing (58±2% and 25±1% respectively). This effect was observed for both low dose and high dose cisplatin concentrations and across the ovarian cell lines (FIG. 1B, SKOV-3; FIG. 1C, OVCAR-8; PEO-1 lines, FIG. 7A, 7B and PEO-6, FIG. 7C). Immunoblot analysis of LB100 pre-treatment in combination with cisplatin in SKOV-3 and OVCAR-8 showed an increased expression of cleaved caspase 3 and cleaved PARP, indicating apoptotic induction as the mechanism of cell death (FIG. 1D). In SKOV-3 cells, LB100 sensitization greatly enhanced the expression of apoptotic factors in combination with an IC₂₅ dose of cisplatin (FIG. 1E).

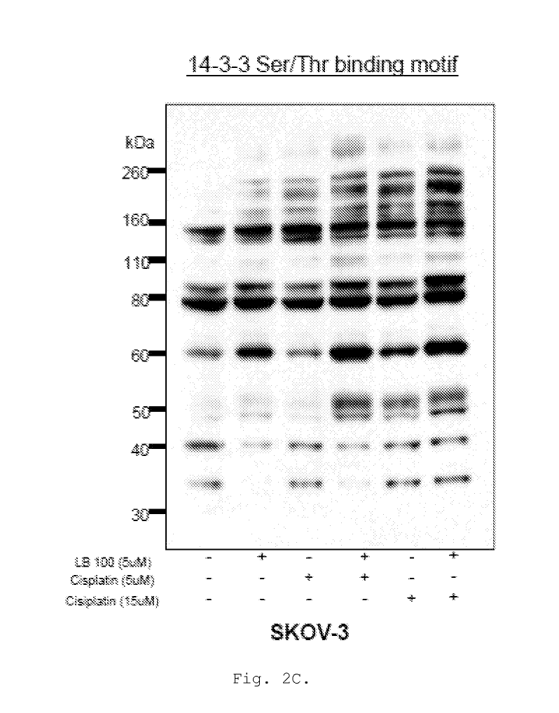

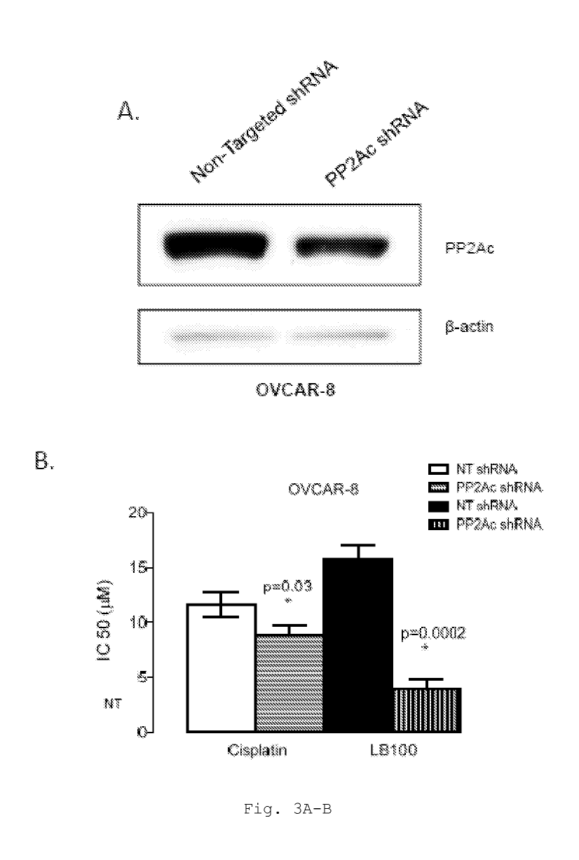

Since pharmacologic inhibition of PP2A via LB100 sensitized ovarian cancer cells to cisplatin, it was investigated whether stable knockdown of expression of the catalytic subunit of PP2A (PP2Ac) might result in the same effect. Stable expression of PP2Ac-specific shRNA in SKOV-3 cells resulted in vastly decreased numbers of viable cells, highlighting that a certain baseline expression of PP2A is essential for cellular viability (Gotz, J. et al. 1998). Conversely, stable knockdown of PP2Ac was achieved in OVCAR-8 cells, with approximately 50% knockdown of PP2Ac expression (FIG. 3A). Cisplatin and LB100 sensitivity was determined for OVCAR-8 PP2Ac shRNA-expressing cells (FIG. 3B). Consistent with the pharmacologic sensitization induced by LB100, PP2Ac knockdown sensitized OVCAR-8 cells to cisplatin compared to non-targeted control. Sensitivity to LB100 was greatly enhanced in the PP2Ac knockdown cells compared to control (LB100 OVCAR-8 NT shRNA IC₅₀=15.7±1.3 μM, LB100 OVCAR-8 PP2Ac shRNA IC₅₀=3.9±0.9 μM, FIG. 3B).

### Example 3. LB100 Induces Constitutive Phosphorylation of Key Mediators in the DNA Damage Response Pathway Allowing Persistent DNA Damage

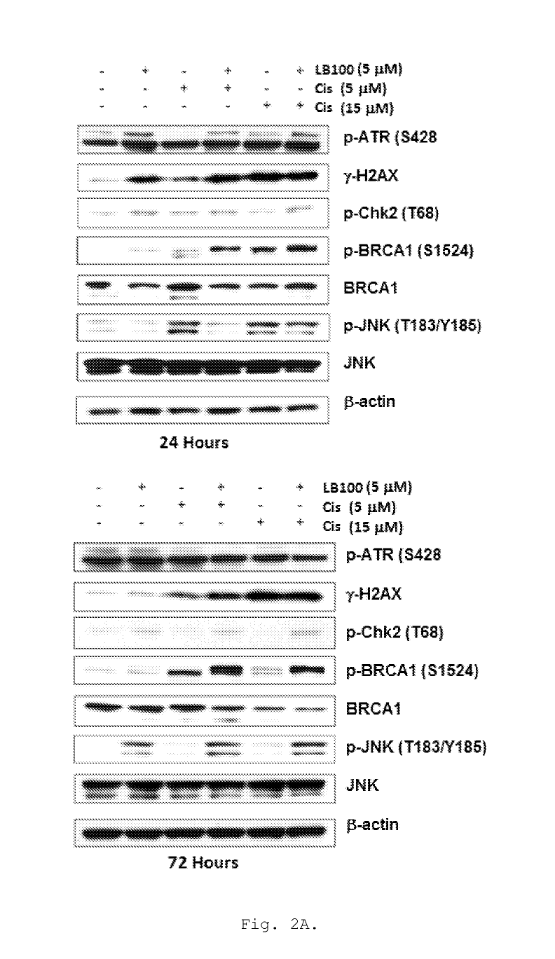

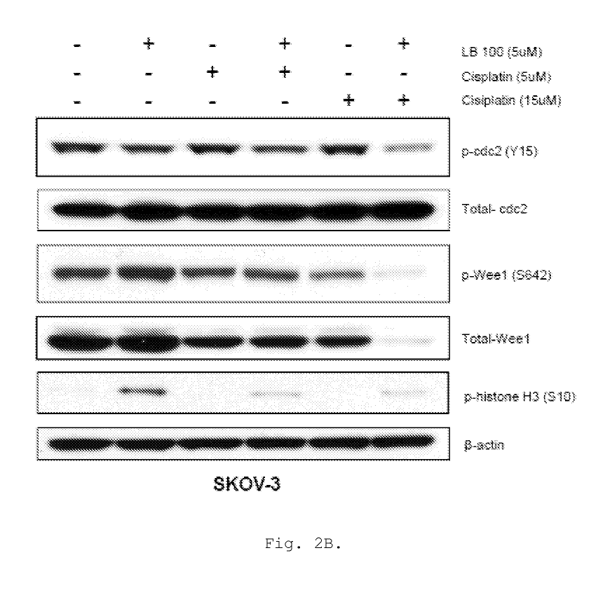

PP2A has been associated with dephosphorylation of γH2AX, Chk2, and BRCA (Chowdhury, D. et al. 2005; Dozier, C. et al. 2004; Carlessi, L. et al. 2010). Persistent expression of γ-H2AX is an indicator of inadequate DNA damage repair (Kinner, A. et al. 2008; Moon, S. H. et al. 2010), and its time-sensitive dephosphorylation is critical for maintaining the chronologic fidelity of repair initiation (Lee, D. H. et al. 2011; Nussenzweig, A. et al. 2006). Furthermore, constitutive phosphorylation of BRCA1 and JNK has been shown to bias the cell towards apoptosis following induction of DNA damage (Martin, S. A. et al. 2005; Mansouri, A. et al. 2003). In order to understand the potential mechanism by which LB100 pre-treatment sensitizes ovarian cancer cells to the effect of cisplatin, the phosphorylation state of these key intermediaries of the DNA damage response pathway following treatment were compared with various combinations of LB100 and cisplatin. Inhibition of PP2A with LB100 both enhanced and prolonged the phosphorylation of γH2AX, Chk2, and BRCA1 at 24 h, and JNK at 72 h (FIG. 2A). This constitutive phosphorylation was not due to marked changes in overall protein level. LB100 plus cisplatin (5 μM) hyperphosphorylated γH2AX, Chk2, and BRCA1 at 24 and 72 h compared to cisplatin (5 μM) alone. For the IC₇₅ dose of cisplatin (15 μM), LB100 pre-treatment led to hyperphosphorylation of Chk2 and BRCA1, compared to cisplatin alone, while expression of γH2AX was similar for both groups. Phosphorylation levels of JNK were greater for cisplatin, compared to LB100 plus cisplatin initially (24 h), but the combination treatment resulted in greater JNK phosphorylation at 72 h compared to both doses of cisplatin alone.

### Example 4. Inhibition of PP2A by LB100 Induces Hyperphosphorylation of Chk1

Chk1 is a central mediator of the DNA damage response and maintains the integrity of the genome by inducing S or G2/M cell cycle arrest and promoting DNA repair. Additionally, the functional integrity of Chk1 is maintained by continuous dephosphorylation of key serine residues such as 5345, by PP2A (Leung-Pineda, V. et al. 2006). In order to assess whether inhibition of PP2A by LB100 could sensitize the DNA damage response pathway by inducing hyperphosphorylation of Chk1 at S345, OVCAR-8 cells were treated with cisplatin for 1 h with or without a 1 h pre-treatment with LB100. The cells were then washed and incubated in media with or without LB100 and allowed to recover for up to 8 h. LB100 significantly increased the phosphorylation of Chk1 at S345 for each time point following cisplatin treatment compared to cells incubated in media alone (FIG. 3D). To confirm whether this differential phosphorylation was due to decreased PP2A function, the same experiment in the stable PP2Ac knockdown OVCAR-8 cells was performed (FIG. 3C). Consistent with the pharmacologic data, decreased expression of PP2Ac resulted in hyperphosphorylation of Chk1 following cisplatin, compared to OVCAR-8 cells stably expressing control, non-targeting shRNA.

### Example 5. Cisplatin-Induced Cell Cycle Checkpoints are Abrogated by LB100, which are Mediated by Changes in Both Weal Expression and Cdc2 Activation

Given the integral interactions between PP2A and numerous cell cycle checkpoint proteins, it was assessed whether LB100 could abrogate cisplatin-induced cell cycle arrest. FACS analysis was performed on SKOV-3 and OVCAR-8 cells at both 24 and 48 h following treatment with various concentrations of both cisplatin and LB100 (Table 2). LB100 treatment alone caused SKOV-3 cells to progress through the G1 stage, resulting in a significantly higher concentration of cells in the G2/M phase. Additionally, this LB100-mediated event was concentration-dependent [Cell fraction in G/2M(%): control (19.4±0.9), LB100 (2 μM) (25.1±0.8), LB100 (10 μM) (32.1±1.6), LB100 (15 μM) (33.9±1.4)]. In agreement with previous reports (Eastman, A. 1999; Sorenson, C. M. et al. 1990), cisplatin induced either slow S-phase progression/arrest (SKOV-3) or G2/M-phase arrest, which appeared over 48 h (OVCAR-8). When each cell line was pre-treated for 1 h with IC₂₅ concentrations of LB100, cell cycle arrest was abrogated at both 24 h and 48 h. In SKOV-3 cells, for example, cisplatin (18 μM) alone resulted in 33±2% of cells in the S-phase while pre-treatment with LB100 (5 μM) resulted in 27±2% of S-phase cells.

TABLE 2
Cell cycle analysis of SKOV-3 and OVCAR8 cells

| | SKOV-3 | | | | OVCAR-8 | | |
|---|---|---|---|---|---|---|---|
| | G₁ (%) | S (%) | G₂/M (%) | | G₁ (%) | S (%) | G₂/M (%) |
| 24 Hour Treatment | | | | | | | |
| Control (PBS) | 58.0 ± 0.9 | 21.1 ± 0.6 | 20.9 ± 0.8 | Control (PBS) | 39.8 ± 0.9 | 40.9 ± 0.5 | 19.3 ± 0.3 |
| LB100 (5 μM) | 51.4 ± 1.5 | 19.7 ± 0.8 | 28.9 ± 1.2 | LB100 (2 μM) | 45.4 ± 1.5 | 37.0 ± 1.6 | 17.6 ± 0.4 |
| Cisplatin (18 μM) | 49.8 ± 2.3 | 33.6 ± 1.8 | 16.6 ± 1.3 | Cisplatin (5 μM) | 4.6 ± 2.0 | 81.9 ± 1.6 | 13.4 ± 0.6 |
| Cisplatin (18 μM) + | 55.1 ± 1.2 | 26.6 ± 2.2 | 18.3 ±2.9 | Cisplatin (5 μM) + | 18.5 ± 0.4 | 59.2 ± 1.0 | 22.3 ± 0.7 |
| LB100 (5 μM) | | | | LB100 (2 μM) | | | |
| Control (PBS) | 49.3 ± 0.3 | 18.7 ± 0.5 | 32.0 ± 0.8 | | | | |
| Cisplatin (2 μM) | 23.0 ± 0.2 | 68.6 ± 0.1 | 8.4 ± 0.3 | | | | |
| Cisplatin (5 μM) | 11.6 ± 0.5 | 75.2 ± 1.0 | 13.5 ± 0.7 | | | | |
| Cisplatin (20 μM) | 67.7 ± 1.2 | 16.0 ± 1.2 | 16.3 ± 0.9 | | | | |
| 48 Hour Treatment | | | | | | | |
| Control (PBS) | 58.0 ± 0.9 | 14.8 ± 0.5 | 19.4 ± 0.9 | Control (PBS) | 60.8 ± 0.9 | 28.0 ± 0.2 | 11.3 ± 0.3 |
| LB100 (5 μM) | 47.2 ± 1.9 | 30.2 ± 3.1 | 25.1 ± 0.8 | LB100 (2 μM) | 55.3 ± 0.4 | 29.9 ± 1.1 | 14.9 ± 1.5 |
| Cisplatin (18 μM) | 40.4 ± 0.8 | 38.4 ± 0.5 | 21.2 ± 1.0 | Cisplatin (5 μM) | 0.85 ± 0.3 | 20.7 ± 2.1 | 78.5 ± 2.2 |
| Cisplatin (18 μM) + | 50.4 ± 4.1 | 24.7 ± 1.6 | 25.0 ± 3.1 | Cisplatin (5 μM) + | 0.98 ± 0.2 | 32.6 ± 0.7 | 66.5 ± 0.5 |
| LB100 (5 μM) | | | | LB100 (2 μM) | | | |
| Control (PBS) | 57.9 ± 0.4 | 22.7 ± 1.2 | 19.4 ± 0.9 | | | | |
| LB100 (2 μM) | 49.8 ± 0.5 | 25.1 ± 0.3 | 25.1 ± 0.8 | | | | |
| LB100 (10 μM) | 36.8 ± 1.8 | 31.1 ± 0.8 | 32.1 ± 1.6 | | | | |
| LB100 (15 μM) | 42.3 ± 1.6 | 23.8 ± 0.2 | 33.9 ± 1.4 | | | | |

Transition into mitosis is critically dependent on the activation state of the cdc2/cyclin B complex (Hermeking, et al. 2006; Reinhardt, H. C. et al. 2009). Cdc2 is negatively regulated by the Wee1 kinase through an inhibitory phosphorylation on Y15 and is positively regulated by the cdc25C phosphatase via dephosphorylation at this same residue. It was assessed whether LB100 induced checkpoint abrogation and cell cycle progression observed in the functional FACS study is due to alterations of checkpoint protein function and/or expression by immunoblotting for p-Wee1 (S642), total Wee1, p-cdc2 (Y15), and total cdc2 in SKOV-3 cells treated for 24 h with PBS (vehicle control), LB100 (5 μM), and cisplatin (5 μM or 15 μM) following 1 h pre-treatment with LB100 (5 μM). LB100 (5 μM) induced hyperphosphorylation of Wee-1 compared to control (FIG. 2B). This differential phosphorylation was not observed when LB100 was added to either concentration of cisplatin. In contrast, Wee1 phosphorylation was nearly absent for the LB100 and cisplatin (15 μM) combination. Total Wee1 expression was also decreased for this treatment group, suggesting that degradation or decreased production resulted in the observed decrease in phosphorylation. Decreased Wee1 expression was correlated with a decrease in p-cdc2 (Y15) for both doses of cisplatin when pre-treated with LB100 as well as LB100 alone, allowing cell cycle progression into mitosis, indicated by the increased expression of p-Histone H3 (Wei, et al. 1998).

### Example 6. LB100 Sensitizes Tumor Cells to Cisplatin In Vivo

The biological efficacy of LB100-induced cisplatin sensitization in an in vivo mouse model of metastatic ovarian carcinoma was assessed. Tumors were established in nude athymic female mice via i.p. injection of SKOV-3 cells transfected with firefly luciferase. Compared to other animal models of ovarian carcinoma, i.p. inoculation better recapitulates the metastatic spread observed in the clinical setting (Hamilton, T. C. et al. 1984). Mice were randomized into four groups [vehicle (PBS) control (n=4), LB100 (1.5 mg/kg) (n=5), cisplatin (1.5 mg/kg)(n=5), and LB100 (given 1 h prior to cisplatin)+cisplatin (n=5)] and treated six times, with drugs administered every other day, starting from four days after tumor inoculation. Following the final treatment, mice were observed until pre-determined health concerns necessitated euthanization. Dose and treatment schedules were determined from biologic profiles of each agent determined in previous studies (Wei, D. et al. 2013; Lu, J. et al. 2009; Mabuchi, S. et al. 2009) and disease progression was monitored by BLI.

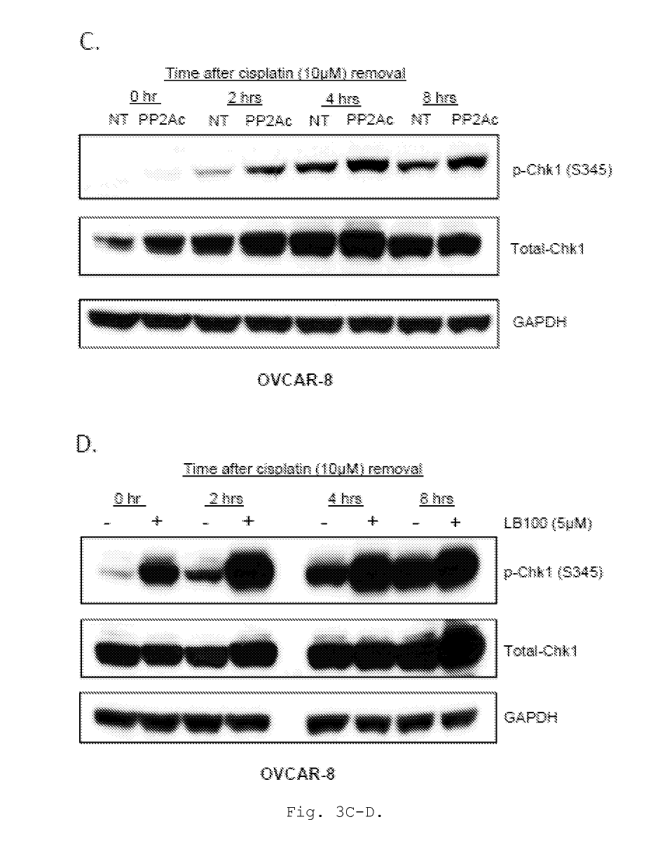

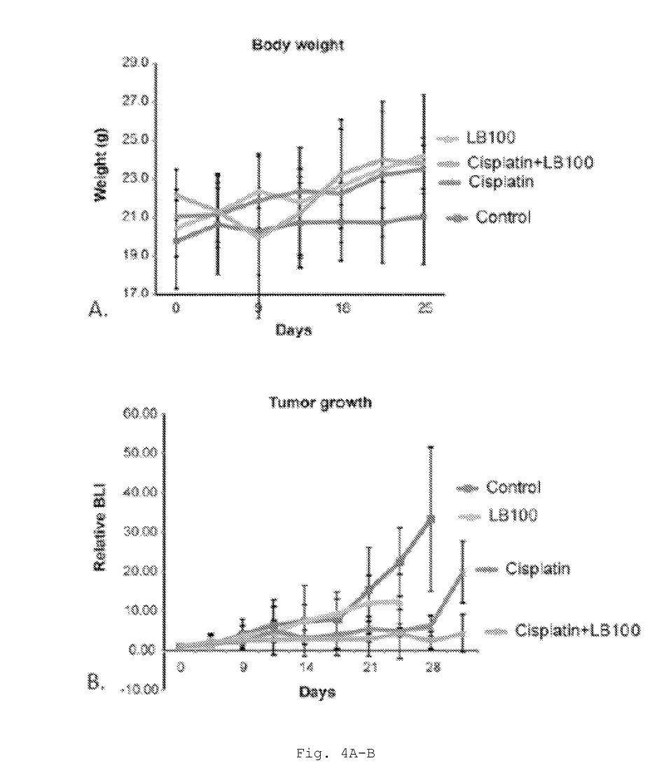

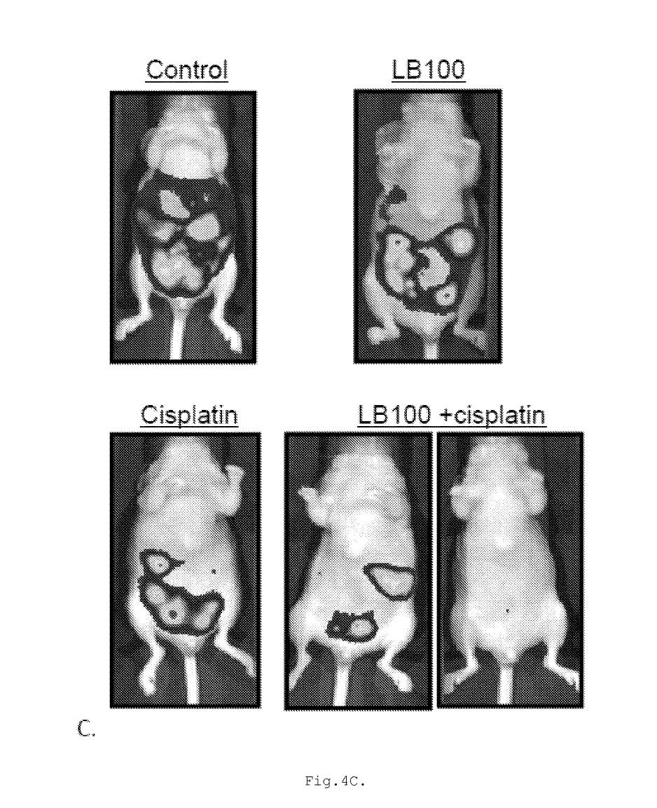

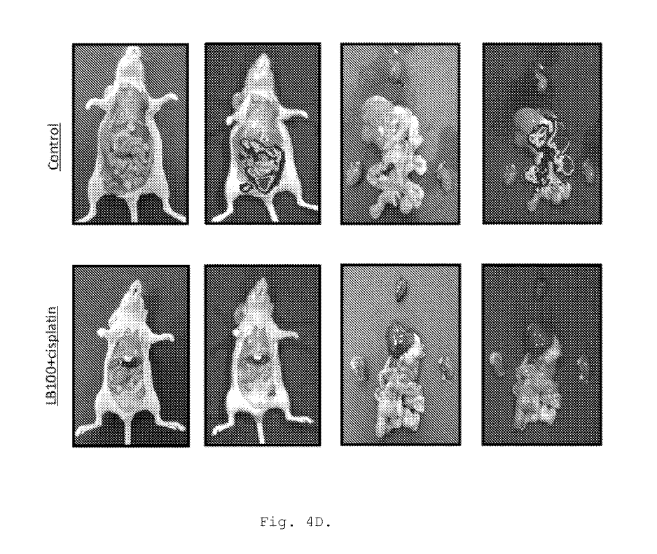

There was no significant difference in mean body weight amongst the four treatment groups, indicating minimal toxicity of the compounds (FIG. 4A). LB100 alone did not alter tumor growth, as assessed by BLI (FIG. 4B, 4C). On the other hand, cisplatin (relative intensity 5.0±1.6) and the combination of cisplatin and LB100 (4.1±6.3) significantly delayed disease progression by day 25 compared to vehicle (22.4.±8.7) and LB100 alone (12.4±6.7). By day 35, the combination treatment significantly delayed tumor progression compared to cisplatin alone (4.3±4.7 vs. 19.8±7.9, p=0.03). By day 25 and day 28, massive ascites developed in the LB100 and control group respectively, necessitating euthanasia. Ex vivo analysis confirmed that the BLI signals were originating from the tumors (FIG. 4D).

Next, it was assessed whether the same molecular mechanisms observed in the in vitro studies were involved in the LB100-induced sensitization of intraperitoneal tumors to cisplatin. Consistent with the in vitro findings, LB100 alone induced hyperphosphorylation of BRCA1, Wee1, Chk1, γH2AX (FIG. 4E). BRCA1 and Chk-1 phosphorylation was further enhanced in the combination treatment with cisplatin, compared to cisplatin alone. While not as robust compared to in vitro findings, the combination treatment resulted in decreased phosphorylation of Wee-1 at S642 compared to cisplatin alone, indicating progression of cell cycle pass the G2/M checkpoint. Combination treatment resulted in hyperphosphorylation of Chk1 and enhanced caspase 3 cleavage, indicating sensitization of cisplatin induced apoptosis.

### Example 7. Administration of LB-100 in Combination with an Anti-Cancer Agent

An amount of compound LB-100 in combination with an anti-cancer agent is administered to a subject afflicted with ovarian cancer. The amount of the compound and anti-cancer agent is effective to treat the ovarian cancer

An amount of compound LB-100 in combination with cisplatin or doxorubicin is administered to a subject afflicted with ovarian cancer. The amount of the compound and the cisplatin or doxorubicin is effective to treat the ovarian cancer.

An amount of compound LB-100 in combination with an anti-cancer agent is administered to a subject afflicted with ovarian cancer that is resistant to the anti-cancer agent or at least one other anti-cancer agent. The amount of the compound and anti-cancer agent is effective to treat the ovarian cancer

An amount of compound LB-100 in combination with cisplatin or doxorubicin is administered to a subject afflicted with ovarian cancer that is resistant to the anti-cancer agent or at least one other anti-cancer agent. The amount of the compound and the cisplatin or doxorubicin is effective to treat the ovarian cancer.

An amount of compound LB-100 in combination with an anti-cancer agent is administered to a subject afflicted with ovarian cancer. The amount of the compound is effective to reduce the likelihood of the ovarian cancer developing resistance to the anti-cancer agent.

An amount of compound LB-100 in combination with cisplatin or doxorubicin is administered to a subject afflicted with ovarian cancer. The amount of the compound is effective to reduce the likelihood of the ovarian cancer developing resistance to the cisplatin or doxorubicin.

An amount of compound LB-100 in combination with an anti-cancer agent is administered to a subject afflicted with ovarian cancer. The amount of the compound is effective to enhance the anti-cancer activity of the anti-cancer agent.

An amount of compound LB-100 in combination with cisplatin or doxorubicin is administered to a subject afflicted with ovarian cancer. The amount of the compound is effective to enhance the anti-cancer activity of the cisplatin or doxorubicin.

### Example 8. Administration of LB-100 Analogs in Combination with an Anti-Cancer Agent

An amount of any one of the compounds of the present invention, which are analogs of LB-100, in combination with an anti-cancer agent is administered to a subject afflicted with ovarian cancer. The amount of the compound and anti-cancer agent is effective to treat the ovarian cancer

An amount of any one of the compounds of the present invention, which are analogs of LB-100, in combination with cisplatin or doxorubicin is administered to a subject afflicted with ovarian cancer. The amount of the compound and the cisplatin or doxorubicin is effective to treat the ovarian cancer.

An amount of any one of the compounds of the present invention, which are analogs of LB-100, in combination with an anti-cancer agent is administered to a subject afflicted with ovarian cancer that is resistant to the anti-cancer agent or at least one other anti-cancer agent. The amount of the compound and anti-cancer agent is effective to treat the ovarian cancer

An amount of any one of the compounds of the present invention, which are analogs of LB-100, in combination with cisplatin or doxorubicin is administered to a subject afflicted with ovarian cancer that is resistant to the anti-cancer agent or at least one other anti-cancer agent. The amount of the compound and the cisplatin or doxorubicin is effective to treat the ovarian cancer.

An amount of any one of the compounds of the present invention, which are analogs of LB-100, in combination with an anti-cancer agent is administered to a subject afflicted with ovarian cancer. The amount of the compound is effective to reduce the likelihood of the ovarian cancer developing resistance to the anti-cancer agent.

An amount of any one of the compounds of the present invention, which are analogs of LB-100, in combination with cisplatin or doxorubicin is administered to a subject afflicted with ovarian cancer. The amount of the compound is effective to reduce the likelihood of the ovarian cancer developing resistance to the cisplatin or doxorubicin.

An amount of any one of the compounds of the present invention, which are analogs of LB-100, in combination with an anti-cancer agent is administered to a subject afflicted with ovarian cancer. The amount of the compound is effective to enhance the anti-cancer activity of the anti-cancer agent.

An amount of any one of the compounds of the present invention, which are analogs of LB-100, in combination with cisplatin or doxorubicin is administered to a subject afflicted with ovarian cancer. The amount of the compound is effective to enhance the anti-cancer activity of the cisplatin or doxorubicin.

### DISCUSSION

Inhibition of PP2A by the novel inhibitors LB-100 and LB-102 and other structural homologs of these compounds have been shown to result in increased phosphorylation of Akt (Lu et al. 2009; U.S. Pat. No. 80,858,268). Phosphorylation of Akt leads to its activation, which in turn increases the phosphorylation of several proteins affecting mitochondrial function and mediating cell death (Tsang et al. 2005).

Recent pre-clinical studies have shown that pharmacologic and genetic inhibition of PP2A sensitizes CNS and pancreatic cancers to radiation and DNA damaging chemotherapy (Wei, D. et al. 2013; Lu, J. et al. 2009; Zhang, C. et al. 2010). Unlike kinase inhibitors, however, few phosphatase inhibitors are undergoing pre-clinical and clinical investigations. The aim of this study was to assess whether LB100, a small-molecular inhibitor of PP2A that is currently undergoing a phase 1 trial, can sensitize pre-clinical models of ovarian cancer to cisplatin. The results contained herein show that pre-treatment with LB-100 enhances cisplatin-induced apoptosis for various ovarian cancer cells in vitro and specifically for SKOV-3 cells in-vivo. This effect was observed for both low (IC₂₅) and high (IC₇₅) doses of cisplatin and was correlated with constitutive phosphorylation of key DNA damage response proteins leading to persistent DNA damage and abrogation of cell cycle arrest, culminating in apoptosis.

The majority of ovarian cancers harbor inactivating mutations of p53. Since p53 orchestrates the G1 to S phase cell cycle check point, cancer cells with aberrant p53 function depend onG2/M arrest for maintaining genomic integrity following DNA damaging therapy (Yarden, R. I. et al. 2002. Entry from G2 into mitosis depends on the activation and nuclear localization of Cdc2/cyclin B, which is negatively regulated by Wee1 and Chk1 kinase and positively regulated by Cdc25C phosphatase. As such, cancer cells that are resistant to DNA damage often induce the overexpression and function of G2/M checkpoint kinases in response to genotoxic stress, and inhibition or downregulation of Wee1 and Chk1 has been shown to sensitize cells to platinum compounds (Pouliot, L. M. et al. 2012). Specific kinase inhibitors have clinical limitations, however, since resistant cells possess alternate pathways that can circumvent inhibition (Lovly, C. M. et al. 2014). On the other hand, ubiquitous Ser/Thr phosphatases such as PP2A are extensively involved in regulation of the DNA response pathway and potentially allow manipulation of multiple signaling pathways through the use of a single agent (Wurzenberger, C. et al. 2011).

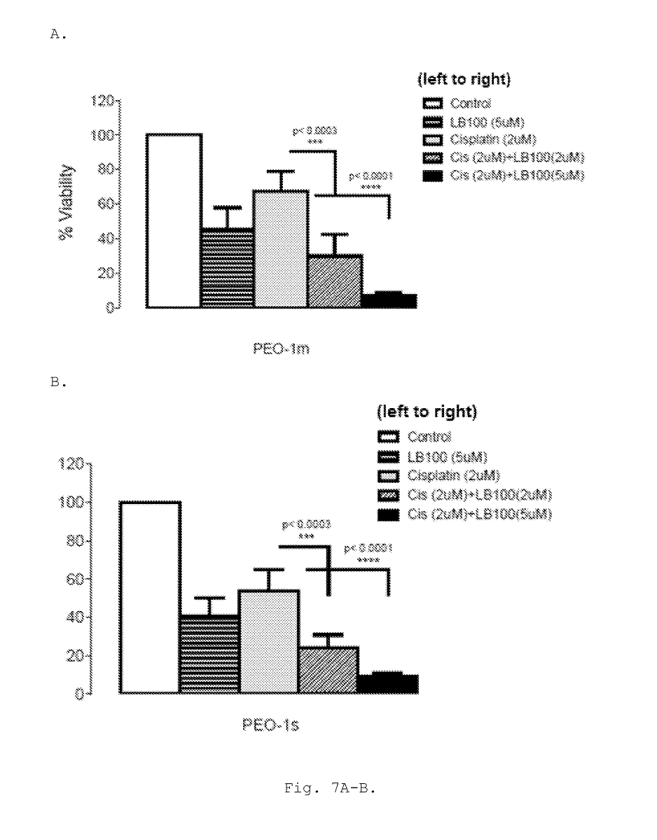

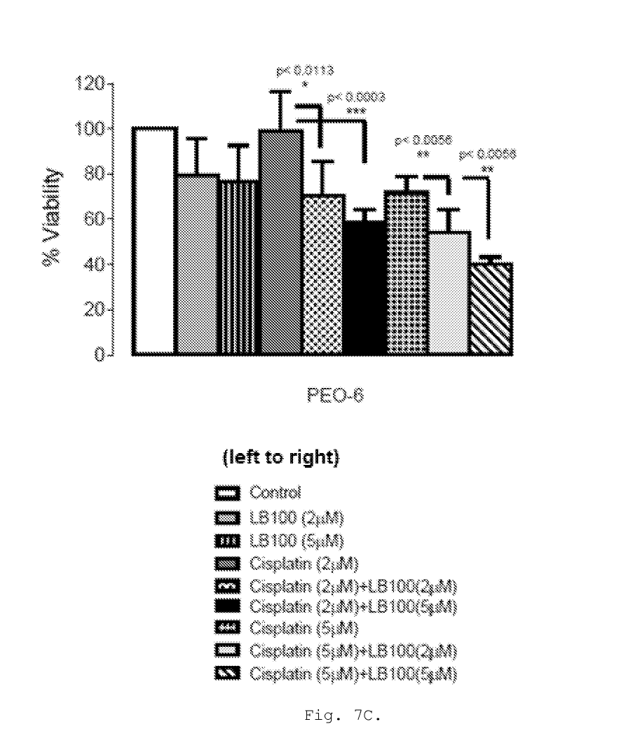

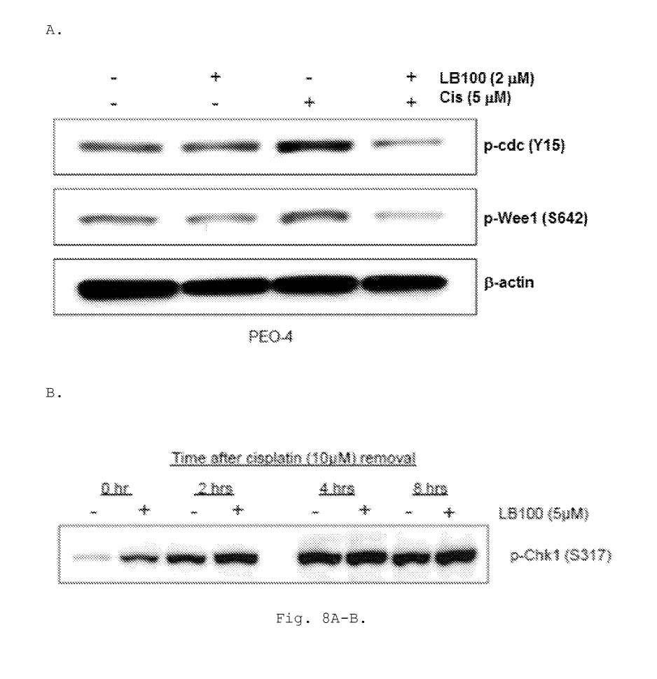

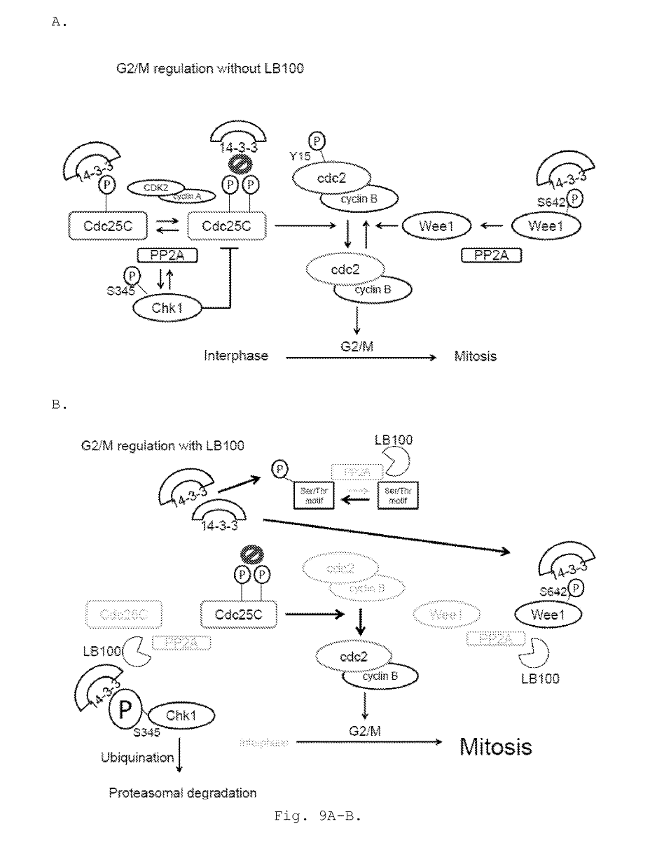

PP2A is an attractive target for DNA damage sensitization for many reasons. Extensive studies in Xenopus have shown that PP2A is induced as part of the DNA damage response and is involved in G2/M arrest (Margolis, S. S. et al. 2006). Thus, inhibition of PP2A leads to aberrant entry into mitosis, resulting in mitotic catastrophe and apoptosis (Castedo, M. et al. 2004). PP2A also regulates Chk1, a critical mediator of DNA damage response (DDR), through a negative feedback loop that maintains Chk1 in a low-activity state during normal cell division, while priming it for rapid response upon DNA damage (Leung-Pineda, V. et al. 2006). This integral relationship is maintained by continuous phosphorylation and dephosphorylation of Chk1 (S345) (Leung-Pineda, V. et al. 2006; Peng, A. et al. 2010). Following DNA damage and DSB formation, ATM/ATR activates Chk1 via phosphorylation at S345, a site negatively regulated by PP2A-mediated dephosphorylation. Constitutive phosphorylation of S345 induces E3 ligase mediated ubiquination and proteasomal degradation, and thus is critical for Chk1 protein stability (Leung-Pineda, V. et al. 2009). Our results show that pharmacologic and genetic inhibition of PP2A by LB100 and PP2Ac shRNA respectively, induces hyperphosphorylation of Chk1 (S345) without altering the phosphorylation state of other serine residues (FIG. 8b). It is possible, therefore, that the LB100-induced cisplatin sensitization may be in part due to deregulation of the negative feedback loop between PP2A and Chk1, rendering Chk1 less effective in the DNA response pathway (FIGS. 9a and 9b).

Through its dephosphorylation activity, PP2A maintains the relative number and distribution of docking sites for chaperone proteins carrying specific phospho-Ser/Thrbinding motifs, such as 14-3-3 and BRCA1 (Kermeking, H. 2003; Mohammad, D. H. et al. 2009). These docking sites exist on a vast array of proteins within the cell, ranging from DNA damage response factors to house-keeping proteins (Snider, N. T. et al. 2014; Reinhardt, H. C. et al. 2013). Docking proteins are vital to cellular homeostasis and cancer biology. For example, the 14-3-3 family of proteins bind to target proteins carrying specific p-Ser/Thr recognition sequences and have been demonstrated to affect the enzymatic activity, DNA-binding activity, sequestration, and protein-protein interactions of these target proteins (Hermeking, H. 2003). In our study, LB100-treated SKOV-3 cells showed widespread increased expression of p-Ser14-3-3 binding motifs compared to control treatment (FIG. 2C), and further showed altered phosphorylation states of proteins specifically known to interact with 14-3-3, such as Wee1 and Chk1. Whether 14-3-3 proteins directly or indirectly affect the activity of these proteins following LB100 and cisplatin combination treatment is yet to be determined. Nonetheless, the results of our study show that LB100-induced modulation of cellular 14-3-3 motifs is correlated with cell cycle progression and enhanced apoptosis. Furthermore, our results also consistently showed that LB00, either alone or in combination with cisplatin, induces hyperphosphorylation of BRCA1 at distinct residues, which was maintained for 72 h. Previous studies have shown that the phosphorylation state of BRCA1 disrupts its interaction with Chk1 and may render cells more sensitive to caspase-3 mediated apoptosis (Martin, S. A. et al. 2005; Yarden, R. I. et al. 2002). BRCA1 contains C-terminal domains (BRCT) that bind to Ser/Thr residues and are integral for BRCA1 mediate DNA damage response. As such, the availability of the BRCT binding domain may be necessary for the proper coordinated response following induction of DNA damage leading to DNA repair (Mohammad, D. H. et al. 2009). LB100-induced deregulation of Ser/Thr motif distribution, as shown in our study, may lead to redistribution of docking-proteins in a way that biases the cisplatin-induced DNA damage response pathway towards mitotic catastrophe and apoptosis.

Given the importance of platinum agents for use in clinical treatment of ovarian cancer and the current paucity of effective treatments, I was hypothesized that LB100 could enhance the effectiveness of cisplatin treatment in ovarian cancer model systems. In vitro studies were performed in various ovarian carcinoma cell lines. LB100-dependent effects on cellular PP2A activity, cytotoxic potentiation, cell cycle modulation, apoptosis and activation of DNA damage signaling and repair pathways were investigated. Additionally, possible additive or synergistic effects of LB100 on cisplatin treatment were determined. In vivo analysis of LB100-induced cisplatin sensitization was conducted in an intraperitoneal (ip) metastatic ovarian cancer model established in athymic nude female mice.

LB100 is an additive or dose-lowering agent that can enhance/maintain the cytotoxic effect of cisplatin without adding undue toxicity. LB100, in combination with docetaxel, is currently being investigated in a phase 1 clinical trial for patients with progressive or metastatic solid tumors who have failed standard treatment, and the initial tolerance seems promising (Chung, V. 2013).

In the context of LB100-induced constitutive phosphorylation of the DNA damage pathway, G2/M arrest abrogation, and modulation of 14-3-3 protein binding motifs observed in this study, it will be of interest to assess the efficacy of LB100 in combination with other preclinical compounds such as inhibitors of Chk1, Wee-1, and PARP1, with and without chemo-radiation. In conclusion, the results contained herein add to the growing literature regarding the efficacy of LB100, and illustrate a potential approach to enhancing cisplatin efficacy during the treatment of ovarian cancer.

The results presented herein showed that LB-100 acts as a chemosensitizer in ovarian cancer xenograft models. This preclinical data provided evidences for a role of LB-100 and PP2A inhibition in ovarian cancer chemotherapy regimen.

### REFERENCES

American Cancer Society. Cancer Facts and FIGS. 2014. 2014.
Armstrong, D. K., et al., Intraperitoneal cisplatin and paclitaxel in ovarian cancer. N Engl J Med, 2006. 354(1): p. 34-43.
Bakhsheshian, J., et al., Bioluminescent imaging of drug efflux at the blood-brain barrier mediated by the transporter ABCG2. Proc Natl Acad Sci USA, 2013. 110(51): p. 20801-6.
Cancer Genome Atlas Research, N., Integrated genomic analyses of ovarian carcinoma. Nature, 2011. 474(7353): p. 609-15.
Carlessi, L., et al., A protein phosphatase feedback mechanism regulates the basal phosphorylation of Chk2 kinase in the absence of DNA damage. Biochim Biophys Acta, 2010. 1803(10): p. 1213-23.
Castedo, M., et al., Cell death by mitotic catastrophe: a molecular definition. Oncogene, 2004. 23(16): p. 2825-37.
Chowdhury, D., et al., gamma-H2AX dephosphorylation by protein phosphatase 2A facilitates DNA double-strand break repair. Mol Cell, 2005. 20(5): p. 801-9.
Chung V, M. A., Phase I study of LB-100 with docetaxel in solid tumors. 2013: ClinicalTrials.gov.
Clemenson, C. and M. C. Marsolier-Kergoat, DNA damage checkpoint inactivation: adaptation and recovery. DNA Repair (Amst), 2009. 8(9): p. 1101-9.
Debernardis, D., et al., p53 status does not affect sensitivity of human ovarian cancer cell lines to paclitaxel. Cancer Res, 1997. 57(5): p. 870-4.
Dozier, C., et al., Regulation of Chk2 phosphorylation by interaction with protein phosphatase 2A via its B′ regulatory subunit. Biol Cell, 2004. 96(7): p. 509-17.
Eastman, A., The mechanism of action of cisplatin: from adducts to apoptosis, in Cisplatin: chemistry and biochemistry of a leading anticancer drug, B. Lippert, Editor. 1999, VHCA, Wiley-VCH. p. 111-134.
Efferth, T., et al., Molecular modes of action of cantharidin in tumor cells. Biochem Pharmacol, 2005. 69(5): p. 811-8.
Efferth, T., et al., Activity of drugs from traditional Chinese medicine toward sensitive and MDR1- or MRP1-overexpressing multidrug-resistant human CCRF-CEM leukemia cells. Blood Cells Mol Dis, 2002. 28(2): p. 160-8.
Eliopoulos, A. G., et al., The control of apoptosis and drug resistance in ovarian cancer: influence of p53 and Bcl-2. Oncogene, 1995. 11(7): p. 1217-28.
Gotz, J., et al., Delayed embryonic lethality in mice lacking protein phosphatase 2A catalytic subunit Calpha. Proc Natl Acad Sci USA, 1998. 95(21): p. 12370-5.
Hall, M. D., et al., Say No to DMSO: Dimethylsulfoxide Inactivates Cisplatin, Carboplatin and Other Platinum Complexes. Cancer Res, 2014.
Hamilton, T. C., et al., Characterization of a xenograft model of human ovarian carcinoma which produces ascites and intraabdominal carcinomatosis in mice. Cancer Res, 1984. 44(11): p. 5286-90.
Hermeking, H., The 14-3-3 cancer connection. Nat Rev Cancer, 2003. 3(12): p. 931-43.
Hermeking, H. and A. Benzinger, 14-3-3 proteins in cell cycle regulation. Semin Cancer Biol, 2006. 16(3): p. 183-92.
Kanchi, K. L., et al., Integrated analysis of germline and somatic variants in ovarian cancer. Nat Commun, 2014. 5: p. 3156.
Kartner, N., J. R. Riordan, and V. Ling, Cell surface P-glycoprotein associated with multidrug resistance in mammalian cell lines. Science, 1983. 221(4617): p. 1285-8.
Kelland, L. R., et al., Ammine/amine platinum(IV) dicarboxylates: a novel class of platinum complex exhibiting selective cytotoxicity to intrinsically cisplatin-resistant human ovarian carcinoma cell lines. Cancer Res, 1992. 52(4): p. 822-8.
Kelland, L. R., et al., Mini-review: discovery and development of platinum complexes designed to circumvent cisplatin resistance. J Inorg Biochem, 1999. 77(1-2): p. 111-5.
Kinner, A., et al., Gamma-H2AX in recognition and signaling of DNA double-strand breaks in the context of chromatin. Nucleic Acids Res, 2008. 36(17): p. 5678-94.
Kovach, J. S., Methods of modulating cell regulation by inhibiting p53. 2012: USA.
Langdon, S. P., et al., Characterization and properties of nine human ovarian adenocarcinoma cell lines. Cancer Res, 1988. 48(21): p. 6166-72.
Lee, D. H. and D. Chowdhury, What goes on must come off: phosphatases gate-crash the DNA damage response. Trends Biochem Sci, 2011. 36(11): p. 569-77.
Leung-Pineda, V., J. Huh, and H. Piwnica-Worms, DDB1 targets Chk1 to the Cul4 E3 ligase complex in normal cycling cells and in cells experiencing replication stress. Cancer Res, 2009. 69(6): p. 2630-7.
Leung-Pineda, V., C. E. Ryan, and H. Piwnica-Worms, Phosphorylation of Chk1 by ATR is antagonized by a Chk1-regulated protein phosphatase 2A circuit. Mol Cell Biol, 2006. 26(20): p. 7529-38.
Lovly, C. M. and A. T. Shaw, Molecular pathways: resistance to kinase inhibitors and implications for therapeutic strategies. Clin Cancer Res, 2014. 20(9): p. 2249-56.
Lu, J., et al., Inhibition of serine/threonine phosphatase PP2A enhances cancer chemotherapy by blocking DNA damage induced defense mechanisms. Proc Natl Acad Sci USA, 2009. 106(28): p. 11697-702.
Mabuchi, S., et al., RAD001 inhibits human ovarian cancer cell proliferation, enhances cisplatin-induced apoptosis, and prolongs survival in an ovarian cancer model. Clin Cancer Res, 2007. 13(14): p. 4261-70.
Madigan, J. P., et al., Regulation of Rnd3 localization and function by protein kinase C alpha-mediated phosphorylation. Biochem J, 2009. 424(1): p. 153-61.
Mamenta, E. L., et al., Enhanced replicative bypass of platinum-DNA adducts in cisplatin-resistant human ovarian carcinoma cell lines. Cancer Res, 1994. 54(13): p. 3500-5.
Mansouri, A., et al., Sustained activation of JNK/p38 MAPK pathways in response to cisplatin leads to Fas ligand induction and cell death in ovarian carcinoma cells. J Biol Chem, 2003. 278(21): p. 19245-56.
Margolis, S. S., et al., Role for the PP2A/B56delta phosphatase in regulating 14-3-3 release from Cdc25 to control mitosis. Cell, 2006. 127(4): p. 759-73.
Markman, M., et al., Second-line platinum therapy in patients with ovarian cancer previously treated with cisplatin. J Clin Oncol, 1991. 9(3): p. 389-93.
Martin, S. A. and T. Ouchi, BRCA1 phosphorylation regulates caspase-3 activation in UV-induced apoptosis. Cancer Res, 2005. 65(23): p. 10657-62.
Martiniova, L., et al., Pharmacologic modulation of serine/threonine phosphorylation highly sensitizes PHEO in a MPC cell and mouse model to conventional chemotherapy. PLoS One, 2011. 6(2): p. e14678.
Mohammad, D. H. and M. B. Yaffe, 14-3-3 proteins, FHA domains and BRCT domains in the DNA damage response. DNA Repair (Amst), 2009. 8(9): p. 1009-17.
Moon, S. H., et al., Wild-type p53-induced phosphatase 1 dephosphorylates histone variant gamma-H2AX and suppresses DNA double strand break repair. J Biol Chem, 2010. 285(17): p. 12935-47.
Mumby, M., PP2A: unveiling a reluctant tumor suppressor. Cell, 2007. 130(1): p. 21-4.
Nussenzweig, A. and T. Paull, DNA repair: tails of histones lost. Nature, 2006. 439(7075): p. 406-7.
Partridge, E., et al., Results from four rounds of ovarian cancer screening in a randomized trial. Obstet Gynecol, 2009. 113(4): p. 775 82.
Peng, A. and J. L. Maller, Serine/threonine phosphatases in the DNA damage response and cancer. Oncogene, 2010. 29(45): p. 5977-88.
Pouliot, L. M., et al., Cisplatin sensitivity mediated by WEE1 and CHK1 is mediated by miR-155 and the miR-15 family. Cancer Res, 2012. 72(22): p. 5945-55.
Reinhardt, H. C. and M. B. Yaffe, Phospho-Ser/Thr-binding domains: navigating the cell cycle and DNA damage response. Nat Rev Mol Cell Biol, 2013. 14(9): p. 563-80.
Reinhardt, H. C. and M. B. Yaffe, Kinases that control the cell cycle in response to DNA damage: Chk1, Chk2, and MK2. Curr Opin Cell Biol, 2009. 21(2): p. 245-55.
Robey, R. W., et al., ABCG2-mediated transport of photosensitizers: potential impact on photodynamic therapy. Cancer Biol Ther, 2005. 4(2): p. 187-94.
Shen, D. W., et al., Cisplatin resistance: a cellular self-defense mechanism resulting from multiple epigenetic and genetic changes. Pharmacol Rev, 2012. 64(3): p. 706-21.
Siegel, R., D. Naishadham, and A. Jemal, Cancer statistics, 2012. CA Cancer J Clin, 2012. 62(1): p. 10-29.
Snider, N. T. and M. B. Omary, Post-translational modifications of intermediate filament proteins: mechanisms and functions. Nat Rev Mol Cell Biol, 2014. 15(3): p. 163-77.
Sorenson, C. M., M. A. Barry, and A. Eastman, Analysis of events associated with cell cycle arrest at G2 phase and cell death induced by cisplatin. J Natl Cancer Inst, 1990. 82(9): p. 749-55.
Taniguchi, T., et al., Disruption of the Fanconi anemia-BRCA pathway in cisplatin-sensitive ovarian tumors. Nat Med, 2003. 9(5): p. 568-74.
Wang, D. and S. J. Lippard, Cellular processing of platinum anticancer drugs. Nat Rev Drug Discov, 2005. 4(4): p. 307-20.
Wong, E. and C. M. Giandomenico, Current status of platinum-based antitumor drugs. Chem Rev, 1999. 99(9): p. 2451-66.
Wei, B. R., et al., Serum S100A6 concentration predicts peritoneal tumor burden in mice with epithelial ovarian cancer and is associated with advanced stage in patients. PLoS One, 2009. 4(10): p. e7670.
Wei, D., et al., Inhibition of protein phosphatase 2A radiosensitizes pancreatic cancers by modulating CDC25C/CDK1 and homologous recombination repair. Clin Cancer Res, 2013. 19(16): p. 4422-32.
Wei, Y., et al., Phosphorylation of histone H3 at serine 10 is correlated with chromosome condensation during mitosis and meiosis in Tetrahymena. Proc Natl Acad Sci USA, 1998. 95(13): p. 7480-4.
Wurzenberger, C. and D. W. Gerlich, Phosphatases: providing safe passage through mitotic exit. Nat Rev Mol Cell Biol, 2011. 12(8): p. 469-82.
Yarden, R. I., et al., BRCA1 regulates the G2/M checkpoint by activating Chk1 kinase upon DNA damage. Nat Genet, 2002. 30(3): p. 285-9.
Zhang, C., et al., A synthetic cantharidin analog for the enhancement of doxorubicin suppression of stem cell-derived aggressive sarcoma. Biomaterials, 2010. 31(36): p. 9535-43.

What is claimed is:

1. A method of treating ovarian cancer in a subject afflicted therewith comprising administering to the subject an effective amount of an anti-cancer agent and an effective amount of a compound having the structure: 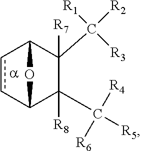
wherein
bond α is present or absent;
R₁ and R₂ together are ═O;
R₁ is OH, O⁻, OR₉, O(CH₂)₁₋₆R₉, SH, S⁻, or SR₉,
wherein R₉ is H, alkyl, alkenyl, alkynyl or aryl;
R₄ is 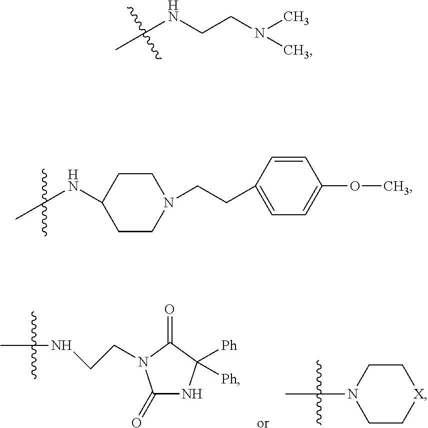
where X is O, S, NR₁₀, N⁺HR₁₀ or N⁺R₁₀R₁₀,
where each R₁₀ is independently H, alkyl, alkenyl, alkynyl, aryl, 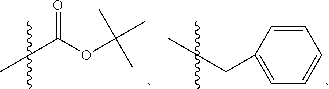
—CH₂CN, —CH₂CO₂R₁₁, or —CH₂COR₁₁,
wherein each R₁₁ is independently H, alkyl, alkenyl or alkynyl;
R₅ and R₆ taken together are ═O;
R₇ and R₈ are each H,
or a salt, zwitterion, or ester thereof,
so as to thereby treat the ovarian cancer in the subject.

2. A method of treating ovarian cancer in a subject afflicted therewith comprising administering to the subject an effective amount of an anti-cancer agent and an effective amount of a compound having the structure: 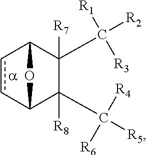
wherein
bond α is present or absent;
R₁ and R₂ together are ═O;
R₃ is OH, O⁻, OR₉, O(CH₂)₁₋₆R₉, SH, S⁻, or SR₉,
wherein R₉ is H, alkyl, alkenyl, alkynyl or aryl;
R₄ is 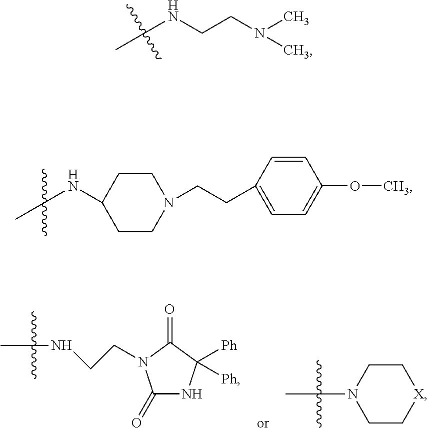
where X is O, S, NR₁₀, N⁺HR₁₀ or N⁺R₁₀R₁₀,
where each R₁₀ is independently H, alkyl, alkenyl, alkynyl, aryl, 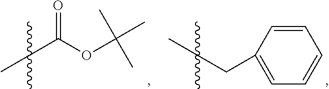
—CH₂CN, —CH₂CO₂R₁₁, or —CH₂COR₁₁,
wherein each R₁₁ is independently H, alkyl, alkenyl or alkynyl;
R₅ and R₆ taken together are ═O;
R₇ and R₈ are each H,
or a salt, zwitterion, or ester thereof,
so as to thereby treat the ovarian cancer in the subject, wherein the ovarian cancer is resistant to the anti-cancer agent or at least one other anti-cancer agent.

3. The method of claim 1 or 2, wherein the ovarian cancer in the subject was previously treated with the anti-cancer agent or at least one other anti-cancer agent.

4. The method of any one of claims 1-3, wherein the amount of the compound and the amount of the anti-cancer agent are each periodically administered to the subject

5. The method of any one of claims 1-3, wherein the amount of the compound and the amount of the anti-cancer agent are administered simultaneously, separately or sequentially.

6. The method of any one of claims 1-3, comprising administering to the subject an effective amount of the compound and subsequently administering to the subject, after an interval comprising at least 1 hour, the anti-cancer agent.

7. The method of any one of claims 1-6, wherein the amount of the compound and the amount of the anti-cancer agent when taken together is more effective to treat the subject than when the anti-cancer agent is administered alone, or when taken together has a greater than additive effect on the ovarian cancer in the subject.

8. The method of any one of claims 1-7, wherein the compound enhances the chemotherapeutic effect of the anti-cancer agent.

9. The method of any one of claims 1-7, wherein the compound chemosensitizes the ovarian cancer to the anti-cancer agent.

10. The method of any one of claim 1-7, wherein the compound reduces the resistance of the ovarian cancer to the anti-cancer agent.

11. The method of any one of claim 1-7, wherein the compound re-sensitizes the ovarian cancer to the anti-cancer agent.

12. A method of reducing the likelihood of a subject afflicted with ovarian cancer developing drug resistance to an anti-cancer agent comprising administering to the subject an effective amount of a compound having the structure: 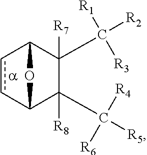
wherein
bond α is present or absent;
R₁ and R₂ together are ═O;
R₃ is OH, O⁻, OR₉, O(CH₂)₁₋₆R₉, SH, S⁻, or SR₉,
wherein R₉ is H, alkyl, alkenyl, alkynyl or aryl;
R₄ is 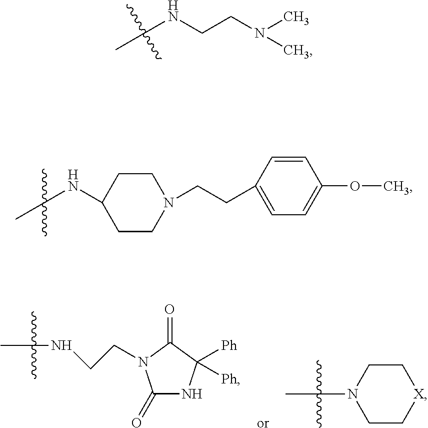
where X is O, S, NR₁₀, N⁺HR₁₀ or N⁺R₁₀R₁₀,
where each R₁₀ is independently H, alkyl, alkenyl, alkynyl, aryl, 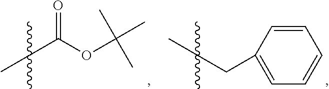
—CH₂CN, —CH₂CO₂R₁₁, or —CH₂COR₁₁,
wherein each R₁₁ is independently H, alkyl, alkenyl or alkynyl;
R₅ and R₆ taken together are ═O;
R₇ and R₈ are each H,
or a salt, zwitterion, or ester thereof,
and administering an effective amount of the anti-cancer agent so as to thereby reduce the likelihood of the subject afflicted with the ovarian cancer developing drug resistance to the anti-cancer agent.

13. The method of claim 12, wherein the ovarian cancer in the subject was previously treated with the anti-cancer agent or at least one other anti-cancer agent.

14. The method of any one of claims 1-13, wherein the amount of compound administered is 0.05-0.25 mg/kg/day, 0.1-0.15 mg/kg/day, 0.2-0.25 mg/k g/day, 7.5-15 mg/day, 7.5-12.5 mg/day, or 10-15 mg/day.

15. The method of any one of claims 1-13, wherein the amount of anti-cancer agent administered is 0.1-0.3 mg/kg/day, 0.1-0.15 mg/kg/day, 0.225-0.275 mg/kg/day, 5-20 mg/day, 5-10 mg/day, or 12.5-17.5 mg/day.

16. The method of any one of claims 1-15, wherein the anti-cancer agent is a platinum-based anti-cancer agent.

17. The method of claim 16, wherein the platinum-based anti-cancer agent is cisplatin, carboplatin, oxaliplatin, satraplatin, picoplatin, nedaplatin, triplatin or lipoplatin.

18. The method of claim 17, wherein the platinum-based anti-cancer agent is cisplatin.

19. The method of any one of claims 1-15, wherein the anti-cancer agent is an anthracycline anti-cancer agent.

20. The method of claim 19, wherein the anthracycline anti-cancer agent is doxorubicin, daunorubicin, epirubicin, idarubicin, or valrubicin.

21. The method of claim 20, wherein the anthracycline anti-cancer agent is doxorubicin.

22. The method of any one of claims 1-21, wherein the compound has the structure 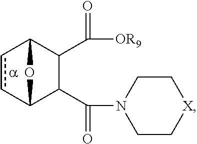
wherein
bond α is present or absent;
R₉ is present or absent and when present is H, alkyl, alkenyl, alkynyl or phenyl; and
X is O, NR₁₀, NH⁺R₁₀ or N⁺R₁₀R₁₀,
where each R₁₀ is independently H, alkyl, substituted alkyl, alkenyl, substituted alkenyl, alkynyl, substituted alkynyl, aryl, 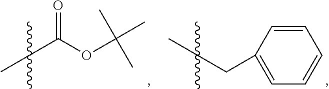
—CH₂CN, —CH₂CO₂R₁₂, or —CH₂COR₁₂,
where R₁₂ is H or alkyl,
or a salt, zwitterion or ester thereof.

23. The method of any one of claims 1-21, wherein the compound has the structure 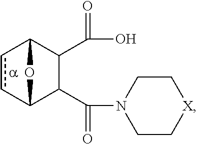
wherein
bond α is present or absent;
X is O or NR₁₀,
where each R₁₀ is independently H, alkyl, substituted alkyl, alkenyl, substituted alkenyl, alkynyl, substituted alkynyl, aryl, 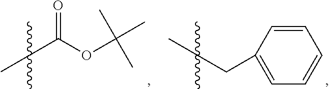
—CH₂CN, —CH₂CO₂R₁₂, or —CH₂COR₁₂,
where R₁₂ is H or alkyl,
or a salt, zwitterion or ester thereof.

24. The method of any one of claims 1-21, where in the compound has the structure 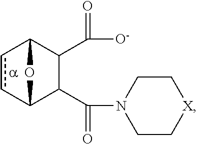
wherein
bond α is present or absent;
X is O or NH⁺R₁₀,
where R₁₀ is H, alkyl, substituted alkyl, alkenyl, substituted alkenyl, alkynyl, substituted alkynyl, aryl, 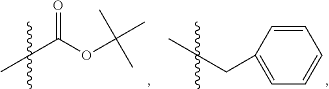
—CH₂CN, —CH₂COR₁₂, or —CH₂COR₁₂,
where R₁₂ is H or alkyl,
or a salt, zwitterion or ester thereof.

25. The method of claim 24, wherein the compound has the structure 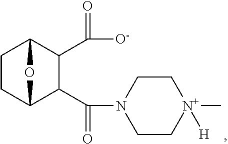 [SMILES: C[NH+]1CCN(C(=O)C2C(C(=O)[O-])[C@H]3CC[C@@H]2O3)CC1]
or a salt or ester thereof.


SEQUENCE LISTING (2 sequences)

SEQ ID NO: 1 - DNA, 21 bp, Artificial Sequence
  PP2AC-shRNA target sequence
tggaacttgacgatactctaa

SEQ ID NO: 2 - DNA, 21 bp, Artificial Sequence
  Primer target sequence
ctggttacgaagcgaatcctt